(19) United States
(12) Patent Application Publication
(10) Pub. No.: US 2026/0207548 A1
(43) Pub. Date: Jul. 23, 2026

(54) METHODS AND COMPOSITIONS FOR THE TREATMENT OF VASCULAR ANOMALIES

(71) Applicant: THE CHILDREN'S MEDICAL CENTER CORPORATION, Boston, MA (US)

(72) Inventors: Joyce E. BISCHOFF, Jamaica Plain, MA (US); Annegret HOLM, Boston, MA (US)

(73) Assignee: THE CHILDREN'S MEDICAL CENTER CORPORATION, Boston, MA (US)

(21) Appl. No.: 19/137,677

(22) PCT Filed: Dec. 11, 2023
(86) PCT No.: PCT/US2023/083306

§ 371 Date: Jun. 11, 2025

Related U.S. Application Data
(60) Provisional application No. 63/432,110, filed on Dec. 13, 2022.

(51) Int. Cl. A61K 31/366; A61K 31/138; A61K 31/165; A61K 31/235; A61K 31/40; A61K 31/405; A61K 31/439; A61K 31/4418; A61K 31/47; A61K 31/505; A61K 31/5377; A61K 31/573; A61K 31/58; A61P 35/00

(52) U.S. Cl. CPC A61K 31/366; A61K 31/138; A61K 31/165; A61K 31/235; A61K 31/40; A61K 31/405; A61K 31/439; A61K 31/4418; A61K 31/47; A61K 31/505; A61K 31/5377; A61K 31/573; A61K 31/58; A61P 35/00

(57) ABSTRACT

Described herein are methods and compositions related to the use of statins to treat vascular anomalies.

### CROSS-REFERENCE TO RELATED APPLICATIONS

This application claims benefit under 35 U.S.C. § 119(e) of U.S. Provisional Application No. 63/432,110 filed Dec. 13, 2022, the contents of which are incorporated herein by reference in their entirety.

### GOVERNMENT SUPPORT

This invention was made with government support under Grant No. HL096384-11 awarded by the National Institutes of Health. The government has certain rights in the invention.

### TECHNICAL FIELD

The technology described herein relates to the treatment of vascular anomalies, e.g., infantile hemangioma.

### BACKGROUND

Vascular anomalies can result in malformed vasculature or proliferation of vascular tumors, sometimes causing a serious threat to a patient's health, as well as aesthetic concerns dependent upon location. For example, infantile hemangioma (IH) is a common childhood tumor composed of disorganized blood vessels and immature cells. IH results from a disruption of neonatal vasculogenesis, de novo formation of vessels from progenitor cells, angiogenesis, and a sprouting of new vessels from pre-existing vasculature. Although benign and usually a harmless tumor, some IH deform or destroy facial features and cause functional impairment such as obstructing vision or impairing breathing. Forty to eighty percent result in permanent cutaneous residua, which can be disfiguring. Corticosteroids were the traditional first-line therapy, however, the adverse effects are numerous (Boon et al., Plast Reconstr Surg 1999 104:1616-1623) and approximately 16% of hemangiomas do not respond (Bennett et al., Arch Dermatol 2001 137:1208-1213). Propranolol was introduced as a treatment for IH (Leaute-Labreze et al., NEJM 2008 358:2649-2651; Siegfried et al., NEJM 2008 359:2846; and Leaute-Labreze et al., NEJM 2015 372:735-46 (PMID 25693013)). However, its use is not without risks, and not all tumors respond (Frieden and Drolet. Pediatr Dermatol 2009 26:642-644). Concerning side effects of propranolol are due to its antagonistic effect on β-adrenergic receptors and include sleep disorder, bronchospasm, bradycardia, hypotension, and hypoglycemia. More effective therapies could improve outcomes and/or shorten treatment duration, thereby reducing the risk of adverse side effects.

### SUMMARY

The technology described herein is directed to methods and compositions relating to the treatment of vascular anomalies, e.g. hemangiomas. Aspects of the technology described herein are based on the inventors' discovery that propranolol targets the mevalonate pathway in differentiating hemangioma-derived stem cells. Statins known to inhibit the mevalonate pathway, were shown by the inventors to block formation of blood vessels from hemangioma-derived stem cells in vivo.

In one aspect of any of the embodiments, described herein is a method of treating a vascular anomaly in a subject in need thereof, the method comprising administering a composition comprising at least one statin to the subject. In one aspect of any of the embodiments, described herein is a composition comprising at least one statin, for use in treating a vascular anomaly.

In one aspect of any of the embodiments, described herein is a combination or kit comprising: at least one statin; and at least one beta-blocker, mTOR inhibitor, and/or steroid. In one aspect of any of the embodiments, described herein is a combination or kit comprising: at least one statin; and at least one beta-blocker, mTOR inhibitor, and/or steroid; for use in treating a vascular anomaly.

In some embodiments of any of the aspects, the statin is selected from the group consisting of: simvastatin; atorvastatin; cerivastatin; fluvastatin; lovastatin; mevastatin; pitavastatin; pravastatin; and rosuvastatin. In some embodiments of any of the aspects, the statin is simvastatin. In some embodiments of any of the aspects, the statin is atorvastatin.

In some embodiments of any of the aspects, the subject is further administered at least one beta-blocker, mTOR inhibitor, and/or steroid.

In some embodiments of any of the aspects, the beta-blocker is selected from the group consisting of: R+ atenolol; atenolol; nadolol; propranolol; R+ propranolol; and timolol. In some embodiments of any of the aspects, the beta-blocker is R+ propranolol.

In some embodiments of any of the aspects, the mTOR inhibitor is selected from the group consisting of: sirolimus; everolimus; temisrolimus; and rapamycin. In some embodiments of any of the aspects, the mTOR inhibitor is rapamycin.

In some embodiments of any of the aspects, the steroid is a corticosteroid. In some embodiments of any of the aspects, the corticosteroid is selected from the group consisting of: dexamethasone; prednisone; prednisolone; triamcinolone; clobetasol propionate; betamethasone valerate; betamethasone dipropionate; and mometasone furoate.

In some embodiments of any of the aspects, the combination, kit, or composition is formulated for oral administration. In some embodiments of any of the aspects, the combination, kit, or composition is a syrup or suspension.

In some embodiments of any of the aspects, the vascular anomaly is selected from the group consisting of: infantile hemangioma, congenital hemangioma, pyogenic granuloma, tufted angioma, kaposiform hemangioendothelioma, epitheloid hemangioendothelioma (EHE), capillary malformations, arteriovenous malformations, venous malformations, lymphatic malformations, complex lymphatic malformations, generalized lymphatic anomaly, central conducting lymphatic anomaly, kaposiform lymphangiomatosis, and Gorham-Stout disease. In some embodiments of any of the aspects, the combination, kit, or composition the vascular anomaly is infantile hemangioma.

### BRIEF DESCRIPTION OF THE DRAWINGS

FIGS. 1A-1H demonstrate that statins inhibit blood vessel formation in a xenograft model of infantile hemangioma. (FIG. 1A) Schematic of IH xenograft model. Patient-derived Hemangioma-derived stem cells (HemSC) isolated from hemangioma specimens from three different infants, designated as HemSC 150A, 147, and 125, were pretreated with 0.5 μM atorvastatin or 5 μM simvastatin or an equivalent DMSO concentration (0.1%) for 24 hours, suspended in Matrigel with 0.25 pM atorvastatin or 2.5 μM simvastatin and an equivalent DMSO concentration (0.1%) and injected into nude mice with 2 implants/mouse. Mice were treated with 0.1-50 mg/kg/d simvastatin and vehicle (max. 11.4% DMSO) twice a day via intraperitoneal injections. The mice were treated with 1, 5, 10 or 15 mg/kg/d atorvastatin, 0.1, 0.5, 1, 5, 10 or 50 mg/kg/d simvastatin or an equivalent volume of PBS with a maximum concentration of DMSO twice a day for 7 days as depicted in the schematic. A significant inhibitory effect on vessel formation was observed at 1 mg/kg/d simvastatin and atorvastatin. (FIG. 1B) Matrigel implants harvested after 7 days of treatment are shown in the top panel. H&E staining indicates reduction of blood vessels in simvastatin treated compared to control mice (middle panels). Anti-human CD31 staining confirmed reduced human vessel formation in simvastatin-treated mice compared to controls (bottom panels). Cell nuclei were stained with DAPI. Scale bars 100 μm. Blood vessels were identified by luminal structures containing 1 or more red blood cells and counted in 5 fields/section, 2 sections/implant from the middle of the implant. Each field was 1331.95×650.7 pm=0.86671 mm2. Treatment with either atorvastatin or simvastatin showed a significant reduction in vessel density at each dosage. Vessel density is expressed in vessels/mm². P values were calculated using ordinary one-way ANOVA. Data show the mean±SEM and were collected from 2 implants in each mouse, leading to an observation sample size of n=28 for vehicle ctr (combined), atorvastatin n=8 (1 mg/kg/d), n=6 (5 mg/kg/d), n=14 (10 mg/kg/d), and n=8 (15 mg/kg/d), as well as simvastatin n=6 (10 mg/kg/d) and n=4 (50 mg/kg/d). (FIG. 1C) Quantification of vessels/mm2 in the H&E-stained sections (left) and human CD31+ vessels/mm2 (right) show a significant reduction in vessel density in the implants of simvastatin-treated mice compared to control mice. (FIG. 1D) Dose response to simvastatin indicates significant inhibition of vessel formation at of 1 mg/kg/day. P values were calculated using one-way ANOVA and the Tukey's multiple comparison test. Data are shown as mean±SD. Data were collected for 2 implants in each mouse, leading to an observation sample size of n=24 for vehicle (combined), n=14 for 10 mg/kg/d, and n=16 for 50 mg/kg/d simvastatin as well as in the dose response n=10 for vehicle, n=10 for 10 mg/kg/d, n=10 for 5 mg/kg/d, n=10 for 1 mg/kg/d, n=10 for 0.5 mg/kg/d, and n=10 for 0.1 mg/kg/d simvastatin. (FIG. 1E) Patient-derived HemSC (n=4) were pretreated with 0.5 μM atorvastatin or vehicle (0.005% DMSO) for 24 hours, suspended in Matrigel with 0.25 μM atorvastatin or an equivalent DMSO concentration (0.0025%) and injected subcutaneously into nude mice with 2 implants/mouse. Mice were treated with 1, 5, 10 or 15 mg/kg/d atorvastatin or an equivalent volume of PBS with a DMSO concentration of max. 0.15% every 12 hours for 7 days. Treatment with atorvastatin leads to a significant reduction in vessel density at each dose. Vessel density is expressed in vessels/mm2. P values are calculated using ordinary one-way ANOVA and the Tukey's multiple comparison test. Data show the mean±SD and were collected for 2 implants in each mouse, leading to an observation sample size of n=22 for vehicle (combined), n=8 (1 mg/kg/d), n=6 (5 mg/kg/d), n=14 (10 mg/kg/d), and n=8 (15 mg/kg/d). (FIG. 1F) A significant reduction of vessel density was observed at 1 mg/kg/d for simvastatin and atorvastatin (*). The human equivalent doses of simvastatin and atorvastatin are shown below. The box highlights the human equivalent dose of simvastatin used in infants with Smith-Lemli-Opitz syndrome (0.5-1 mg/kg/d). (FIGS. 1G, 1H) Blood glucose levels and body weight of mice were unaffected in both simvastatin-treated and control mice.

FIGS. 2A-2E demonstrate that R(+) propranolol educes MVP transcripts in IH-derived HemSC undergoing endothelial differentiation. FIG. 2A Gene ontology analysis of the subontology biological processes of bulk RNA-seq data set from IH-derived HemSC (n=6). FIG. 2D, Volcano plots show an increase in differentially expressed genes on Day 6 compared to Day 4 of HemSC to endothelial differentiation. FIG. 2C, SOX18 is significantly increased on Day 6 compared to Day 4 (n=3). FIG. 2D, Heat map with MVP gene transcripts at Day 4 and Day 6 as well as ABCA1—a negative regulator of the MVP transcription factor SREBP2. FIG. 2E, HemSC induced to undergo endothelial differentiation were treated with R+ propranolol for 2 hours on Day 6, or from Day 2-6, i.e. 4 day treatment. qPCR analyses for three MVP genes—HMGCS1, HMGCR, MVK, and ABCA1, a negative regulator of the MVP transcription factor SREBP2 (n=3).

FIGS. 3A-3C. FIG. 3A: Experimental steps to induce HemSC to endothelial differentiation. VEGF-B at 10 ng/ml added to serum starved HemSC on Day 0 (n=6). Cells were treated ±R(+) propranolol (20 μM) for 2 hours on day 4 and day 6. FIG. 3B: KEGG pathway analysis indicates steroid biosynthesis and axonal guidance are differentially regulated pathways in differentiating HemSC ±R(+) propranolol. FIG. 3C: Overview of MVP genes regulatored by R+ propranolol (heatmap of FIG. 2C).

FIGS. 4A-4E demonstrate that SOX18 regulates the MVP in endothelial cells. (FIG. 4A) ChIP-seq dataset in HUVEC9 demonstrates SOX18 binding sites within the HMGCS1 and HMGCR gene loci which correspond with ENCODE candidate cis-regulatory elements. (FIGS. 4B-4C) A functional role for SOX18 in cholesterol biosynthesis was tested by pharmacological disruption of SOX18 activity with R+ propranolol or the SOX18 inhibitor Sm4. HUVECs were cholesterol depleted by incubation with methyl beta-cyclodextrin (MBCD) for 16 hours, followed by treatment ±R(+) propranolol or ±Sm4 for 16 hours each. Endogenous cholesterol levels were measured by mass spectrometry. (FIGS. 4D, 4E) Overexpression of Ragged Opossum (RaOp), a dominant negative version of SOX18 that is known to disrupt SOX18 activity, decreased immunofluorescent staining of HMGCS1 and HMGCR in HUVECs.

FIG. 5 depicts a flow cytometry plot of HUVECs expressing fluorescently tagged SOX18RaOp sorted into low, intermediate, and high overexpression of RaOP for analysis in FIGS. 4D, 4E.

FIG. 6 depicts a table of patient cells and tissue used.

FIGS. 7A-7E demonstrate that R(+) propranolol reduces the transcriptionally active form of the MVP regulator SREBP2. (FIG. 7A, 7B) Patient-derived HemSC undergoing endothelial differentiation were treated for 2 hours ±R(+) propranolol on Day 6 and analyzed by Western blot with anti-SREBP2 (n=4). ImageJ quantification shows decreased levels of 62 kDa mature SREBP2 in R+ propranolol treated cells. (FIG. 7C, 7D) HemEC transduced with empty vector control lentivirus treated with R+ propranolol and analyzed by WB for SREBP2. Lentiviral knockdown of SOX18 in IH-derived HemEC (n=3) abrogates sensitivity of SREBP2 to R(+) propranolol. (FIGS. 7E-7G) Fold changes in SREBP2 target genes HMGCS1 and HMGCR as well as the SOX18 target gene NOTCH1 in naïve HemEC. HemEC^{shSOX18} and HemEC transduced with empty vector lentivirus, both treated with R+ propranolol normalized to PBS.

FIGS. 8A-8B demonstrate the efficiency of lentiviral knockdown of SOX18 on mRNA (FIG. 8A) and protein (FIG. 8B) measured by qPCR and Western Blot (n=3 biological replicates, 3 independent lentiviral knockdowns of SOX18 for each). HemEC with >70% knockdown of SOX18 were used for experiments in FIGS. 7A-7E.

FIGS. 9A-9G demonstrate that statins inhibit HemSC endothelial differentiation and blood vessel formation in a xenograft model of IH. (FIG. 9A), Simvastatin (1 μM) and atorvastatin (0.1 μM) inhibited VEGF-B induced endothelial differentiation of HemSC (n=3) in vitro as indicated by reduced mRNA levels of EC markers VE-Cadherin and CD31 over the course of 6 days of treatment. R(+) propranolol (20 μM) served as a positive control. (FIG. 9B), Schematic of IH xenograft model. Patient-derived HemSC (n=4) were pre-treated with 5 μM simvastatin or vehicle (0.005% DMSO), suspended in Matrigel with 2.5 μM simvastatin or vehicle (0.0025% DMSO), and injected subcutaneously into nude mice, with 2 implants/mouse. Mice were treated with 0.1-50 mg/kg/d simvastatin and vehicle (max. 11.4% DMSO) twice a day via intraperitoneal injections. (FIG. 9C), Matrigel implants harvested after 7 days of treatment are shown in the top panel. H&E staining indicates reduction of blood vessels in simvastatin treated compared to control mice (middle panels). Anti-human CD31 staining confirmed reduced human vessel formation in simvastatin-treated mice compared to controls (bottom panels). Cell nuclei were stained with DAPI. Scale bars 100 μm. (FIG. 9D) Quantification of vessels/mm2 in the H&E-stained sections (left) and human CD31⁺ vessels/mm² (right) show a significant reduction in vessel density in the implants of simvastatin-treated mice compared to control mice. (FIG. 9E) Dose response to simvastatin indicates significant inhibition of vessel formation as of 1 mg/kg/d. P values were calculated using one-way ANOVA and the Tukey's multiple comparison test. Data are shown as ±SD. Data were collected for 2 implants in each mouse, leading to an observation sample size of n=24 for vehicle (combined), n=14 for 10 mg/kg/d, and n=16 for 50 mg/kg/d simvastatin (FIG. 9D) as well as in the dose response n=10 for vehicle, n=10 for 10 mg/kg/d, n=10 for 5 mg/kg/, n=10 for 1 mg/kg/d, n=10 for 0.5 mg/kg/d, and n=10 for 0.1 mg/kg/d simvastatin (FIG. 9E). (FIGS. 9F, 9G) Blood glucose levels and body weight of mice were unaffected in both simvastatin-treated and control mice.

FIGS. 10A-10F demonstrate cell viability of statin-treated differentiating HemSC. (FIG. 10A) Simvastatin (0.1-1 μM) or atorvastatin (0.01-0.1 μM) had no effect on cell viability over the course of endothelial differentiation for 6 days. (FIG. 10B) Simvastatin and atorvastatin increase LDL receptor mRNA as expected due to HMGCR inhibition. (FIG. 10C) Patient-derived HemSC (n=4) were pretreated with 0.5 μM atorvastatin or vehicle (0.005% DMSO) for 24 hours, suspended in Matrigel with 0.25 μM atorvastatin or an equivalent DMSO concentration (0.0025%) and injected subcutaneously into nude mice with 2 implants/mouse. Mice were treated with 1, 5, 10 or 15 mg/kg/d atorvastatin or an equivalent volume of PBS with a DMSO concentration of max. 0.15% every 12 hours for 7 days. Treatment with atorvastatin leads to a significant reduction in vessel density at each dose. Vessel density is expressed in vessels/mm². P values are calculated using ordinary one-way ANOVA and the Tukey's multiple comparison test. Data show the mean±SD and were collected for 2 implants in each mouse, leading to an observation sample size of n=22 for vehicle (combined), n=8 (1 mg/kg/d), n=6 (5 mg/kg/d), n=14 (10 mg/kg/d), and n=8 (15 mg/kg/d). (FIGS. 10D, 10E) The density of murine blood vessels in the Matrigel implants were unaffected by either maximum dose of simvastatin or atorvastatin compared to vehicle. (FIG. 10F) Stainings of murine lung with anti-human CD31 and human skin with anti-mouse CD31 with respective human and mouse CD31 antibodies used throughout FIG. 4 each show negative staining demonstrating specificity for human or mouse CD31 staining, respectively.

FIG. 11 depicts a table of human equivalent statin doses. A significant reduction of vessel density was observed at 1 mg/kg/d for simvastatin and atorvastatin (*). The human equivalent doses of simvastatin and atorvastatin are shown. The box highlights the human equivalent dose of simvastatin used in infants with Smith-Lemli-Opitz syndrome (0.5-1 mg/kg/d).

FIG. 12 depicts a table of statistics of in vitro differentiation assay with R+ propranolol, simvastatin, and atorvastatin treatment. Summary of statistical analysis of differentiating HemSC treated with R(+) propranolol, simvastatin, and atorvastatin in FIG. 7.

FIGS. 13A-13E demonstrates nuclear co-localization of SOX18 and SREBP2 in proliferating phase and rebounding IH indicate active MVP. (FIGS. 13A-13D) Human age-matched skin, proliferating IH, involuting IH and rebounding IH stained for SREBP2, SOX18, and the human EC-specific lectin UEA1. Cell nuclei are stained with DAPI. SOX18⁺/SREBP2⁺ double positive cell nuclei (arrowheads) are significantly more abundant in proliferating phase, and appear increased rebounding IH, compared to involuting phase IH and age-matched skin controls as quantified in (FIG. 13E). P values were calculated using one-way ANOVA and the Šidák multiple comparison test. Data show the mean±SD. Data represent sample sizes for n=4 age-matched skin controls, n=10 for proliferating and involuting phase IH, and n=2 for rebounding IH. Scale bars 50 μm.

FIGS. 14A-14G. (FIGS. 14A-14D) Single fluorescent channels for each antibody in control skin, proliferating, involuting phase and rebounding IH stained for: SREBP2, SOX18, and the human specific lectin UEA1. Cell nuclei stained with DAPI. Scale bars 50 μm. FIGS. 14E-14G. SOX18 (E-11) antibody validation for specificity in HemEC^{naïve}, HemEC^{empty vector Ctr} and HemEC^{shSOX18} cells. Isotype-matched antibody as well as secondary antibody controls. Scale bars 25 μm.

### DETAILED DESCRIPTION

As demonstrated herein, the inventors have discovered that statins are surprisingly efficacious in blocking hemangioma vessel formation. This finding was demonstrated in infantile hemangioma, but the inventors pose that statins will provide therapeutic effect in other types of vascular anomalies that are responsive to propranolol and/or express the transcription factor SOX18. Additionally, statins inhibit AKT phosphorylation and thereby mTOR signaling, which is a SOX18-independent mechanism of statins. This indicates a broader application in vascular anomalies with TEK and PI3K-AKT-mTOR pathway mutations (esp. venous and lymphatic malformations, or entities within the spectrum of complex lymphatic malformations (esp. generalized lymphatic malformation). Furthermore, statins also have an effect on RAS signaling by inhibiting prenylation of respective proteins. This is therefore a further effect of statins to the spectrum of vascular anomaly entities with mutations in the RAS-RAF-MEK-ERK pathway, (esp. arteriovenous malformations, CCLA, KLA, GSD). Accordingly, statins provide therapeutic activity through both SOX18-dependent and SOX18-independent mechanisms, permitting therapeutic benefit in a broad spectrum of vascular anomalies in which mTOR and ERK signaling is involved.

The inventors have shown that statins target the same pathways as propranolol, although they act on different molecules of those pathways. Thus, the use of statins to treat diseases responsive to propranolol is contemplated herein. Accordingly, the use of statins to treat vascular anomalies, particularly those responsive to propranolol and/or which express the transcription factor SOX18, generally is contemplated herein.

In one aspect of any of the embodiments, described herein is a vascular anomaly in a subject in need thereof, the method comprising administering a composition comprising at least one statin to the subject. In one aspect of any of the embodiments, described herein is at least one statin for use in a method of treating a vascular anomaly.

Vascular anomalies comprise a wide spectrum of rare disorders that are attributed to focal disruption in vascular development processes and are classified according to the International Society for the Study of Vascular Anomalies (ISSVA). See, e.g., the ISSVA Classification of Vascular Anomalies 2018 International Society for the Study of Vascular Anomalies Available on the world wide web at issva.org/classification; which is incorporated by reference herein in its entirety. Two distinct entities are differentiated: vascular tumors and vascular malformations. Vascular tumors are further subdivided into benign, locally aggressive, and malignant tumors. Vascular malformations can occur in each vessel type and are thus classified into capillary, venous, arteriovenous and lymphatic malformations. Moreover, mixed vascular malformations and malformations associated with other anomalies are specified. Non-limiting examples of vascular anomalies include, but are not limited to infantile hemangioma, congenital hemangioma, pyogenic granuloma, tufted angioma, kaposiform hemangioendothelioma, epitheloid hemangioendothelioma (EHE), capillary malformations, arteriovenous malformations, venous malformations, lymphatic malformations, and complex lymphatic malformations (generalized lymphatic anomaly, central conducting lymphatic anomaly, kaposiform lymphangiomatosis and Gorham-Stout disease). The diagnosis and biology of hemangiomas are well known in the art, e.g., as described in Richter et al. Int J Pediatr 2012:645678 (PMID 22611412); Merrow et al. RadioGraphics 2016 36:1494-516 (PMID 27517361); Queisser et al. Circulation Research 2021 129:155-173 (PMID 34166070), Mahady et al. Quant Imaging Med Surg 2015 5:886-97 (PMID 26807370); and Behr et al. AJR 2013 200:414-422 (PMID 23345366); each of which is incorporated by reference herein in its entirety.

In some embodiments of any of the aspects, vascular anomalies are vascular tumors or vascular malformations. Vascular malformations are disorders in which the vasculature is improperly formed, resulting in discolored areas, lesions, masses and benign growths that can be locally aggressive (e.g., arteriovenous malformations) or even malignant (e.g., angiosarcoma), and lesions. In some embodiments, vascular malformations can be proliferative vascular disorders. Non-limiting examples of vascular malformations include slow-flow vascular malformations (e.g. capillary malformation, venous malformation, and lymphatic malformation), fast-flow vascular malformations (e.g. arteriovenous fistula, and arteriovenous malformation), and combined-complex vascular malformations. Methods of diagnosing vascular malformations are well known in the art, see, e.g. Buckmiller et al. Oral Diseases 2010 16:405-418; Flors et al. RadioGraphics 2011 31:1321-1340; Paltiel et al. Radiology 2000 214:747-754; and Werner et al. Eur Arch Otorhiolaryngol 2001 258:141-9; each of which is incorporated by reference herein in its entireties. Vascular anomalies are often treated with targeted medical therapy (ideally based on genetic results of an underlying mutation-“theranostics”), surgical reduction, sclerotherapy and laser, alone or in combination.

In some embodiments, vascular anomalies can be a hemangioma or vascular tumor. As used herein “hemangioma” refers to a benign, usually self-involuting tumor of the endothelial cells of blood vessels. A hemangioma can comprise an increased number of blood vessels and/or capillaries. The vessels and/or capillaries can comprise blood and be connected to the circulatory system. Hemangiomas can occur anywhere in the body, but are typically found in or on the skin, particularly on the face and neck, or in the liver. Hemangiomas can form in utero (e.g. congenital hemangioma) or after birth. Most hemangiomas appear within a few months of birth. Examples of hemangioma include, but are not limited to: infantile hemangioma, congenital hemangioma, kaposiform hemangioendothelioma, infant nevoid hemangioma (“strawberry naevus”), senile hemangioma (“cherry hemangioma”), arteriovenous hemangioma (“cirsoid aneurysm”), and verrucous hemangioma. The diagnosis and treatment of hemangiomas is well known in the art and has been described, for example, in Mulliken, J B; Glowacki, J. Plastic and reconstructive surgery 1982 69 (3): 412-22; Greene, A K. Clinics in plastic surgery 2011 38 (1): 1-5; Ernemann, U. et al. European journal of radiology 2010 75(1): 2-11; and Gupta, A. et al. Clinics in plastic surgery 2011 38(1): 31-44; each of which is incorporated by reference herein in its entireties.

Hemangiomas affecting the skin can be diagnosed by visual examination. Other approaches that can aid a physician in detecting and/or diagnosing hemangioma can include ultrasound, MRI, and/or a biopsy. Hemangiomas can be distinguished from numerous similar conditions by immunohistochemical staining for, e.g. GLUT-1. Markers and symptoms of proliferative vascular disorders, e.g. hemangioma can include, but are not limited to tumors of the endothelial cells of blood vessels; an increased number of blood vessels and/or capillaries; discoloration of the skin; GLUT-1 expression; hemosiderin pigmentation; ulceration; and bleeding.

While many hemangiomas will regress without medical intervention, the location and extent of some cases can be harmful or dangerous to the subject's health, e.g. interfere with breathing or vision, or induce bone erosion, high-output heart failure, ulceration, and/or raise the possibility of significant cosmetic injury. Symptoms and complications of hemangioma can include, but are not limited to, ulcerations (break down of the hemangioma), bleeding, occlusion, amblyopia (if the hemangioma is near or in the eye), psychosocial complications, alteration of the subject's appearance, attention and malicious reactions from others, and PHACES syndrome (in the case of segmented hemangiomas of the head and neck). Treatments for hemangioma can include, but are not limited to, oral or topical beta blockers as first line therapy, oral corticosteroid or sirolimus therapy, and surgical removal as well as laser therapy. Hemangiomas of the vertebrae are typically treated with radiation, surgical removal, and/or embolization. The methods and compositions described herein can be combined with any known treatment for hemangioma.

As used herein, “statins” refers to a class of drugs also known as HGM-CoA reductase inhibitors that are known to lower cholesterol. Statins are well known in the art. In some embodiments of any of the aspects, the at least one statin is selected from the group consisting of simvastatin; atorvastatin; cerivastatin; fluvastatin; lovastatin; mevastatin; pitavastatin; pravastatin; and rosuvastatin. In some embodiments of any of the aspects, the at least one statin is selected from the group consisting of simvastatin; atorvastatin; fluvastatin; lovastatin; mevastatin; pitavastatin; pravastatin; and rosuvastatin.

In some embodiments of any of the aspects, the at least one statin comprises simvastatin. In some embodiments of any of the aspects, the at least one statin comprises atorvastatin. In some embodiments of any of the aspects, the at least one statin comprises fluvastatin. In some embodiments of any of the aspects, the at least one statin comprises lovastatin. In some embodiments of any of the aspects, the at least one statin comprises mevastatin. In some embodiments of any of the aspects, the at least one statin comprises pitavastatin. In some embodiments of any of the aspects, the at least one statin comprises pravastatin. In some embodiments of any of the aspects, the at least one statin comprises rosuvastatin.

In some embodiments of any of the aspects, the at least one statin consists of simvastatin. In some embodiments of any of the aspects, the at least one statin consists of atorvastatin. In some embodiments of any of the aspects, the at least one statin consists of fluvastatin. In some embodiments of any of the aspects, the at least one statin consists of lovastatin. In some embodiments of any of the aspects, the at least one statin consists of mevastatin. In some embodiments of any of the aspects, the at least one statin consists of pitavastatin. In some embodiments of any of the aspects, the at least one statin consists of pravastatin. In some embodiments of any of the aspects, the at least one statin consists of rosuvastatin.

As described herein, the inventors have found that statins block hemangioma blood vessel formation in vivo by a mechanism distinct from the targets of existing therapies such as beta-blockers, mTOR inhibitors, and/or steroids. Accordingly, statins can be used in combination with other therapeutic compositions to provide additive and/or synergistic effects.

In some embodiments of any of the aspects, the subject is further administered at least one beta-blocker, mTOR inhibitor, and/or steroid.

As used herein, “beta-blocker” refers to a chemical which inhibits or blocks the activity of one or more beta-adrengenic receptors. Some beta-blockers antagonize one specific subtype of beta-adrenergic receptors (e.g., a beta-1 selective beta blocker which selectively antagonizes the beta-1 adrenergic receptor), whereas other beta-blockers are non-selective. In some embodiments, a beta-blocker can inhibit the effect of, e.g., noradrenaline or norepinephrine on one or more beta-adrengenic receptors. In context of the technology described herein, the term “beta-blocker” refers to all types of antagonists or inhibitors of beta-adrenergic receptors, regardless of whether the beta-blocker antagonizes one, two or more beta-adrenergic receptors and regardless of whether they affect other processes. Examples of beta-blockers include, but are not limited to: acebutolol, alprenolol, atenolol, betaxolol, bisoprolol, bopindolol, bucindolol, butaxamine, carteolol, carvedilol, celiprolol, esmolol, labetalol, levobunolol, medroxalol, metipranolol, metoprolol, nadolol, nebivolol, nadolol, oxprenolol, penbutolol, pindolol, propafenone, propranolol, sotalol, timolol, eucommia bark, ICI-118,551, and SR59230A. As used herein, the term “beta-blocker” can include, but is not limited to, the following generic and brand name beta-blockers: R+ propranolol, HEMANGEOL (oral propranolol) SOTACOR™, BETAPACE™ (sotalol), NOVO-TIMOL™, APO-TIMOL™, BLOCADREN™ (timolol), BREVIBLOC™ (esmolol), CARTROL™ (carteolol), COREG™ (carvedilol), CORGARD™ (nadolol), INDERAL™ (propranolol), INDERAL-LA™, APOPROPRANOLOL™ (propranolol), KERLONE™ (betaxolol), LEVATOL™ (penbutolol), BETALOC™, LOPRESSOR™ NOVOMETOPROL™ (metoprolol), NORMODYNE™ (labetalol), SECTRAL™ (acebutolol), TENORMIN™, NOVO-ATENOL™ (atenolol), TOPROL-XL™ (metoprolol), TRANDATE™ (labetalol), NOVOPINDOL™, VISKEN™ (pindolol), ZEBETA™ (bisoprolol), TRASICOR™ (oxprenolol), APOATENOLOL™ (atenolol), and MONITAN™ and SECTRAL™ (acebutolol). In some embodiments, a beta-blocker can be atenolol (e.g. a compound having the structure of Formula I), nadolol (e.g. a compound having the structure of Formula II), propranolol (e.g. a compound having the structure of Formula III), or timolol (e.g. a compound having the structure of Formula IV). Methods of synthesizing beta-blockers are well known in the art and such compounds are also commercially available, e.g. timolol (Cat. No. T6394, Sigma-Aldrich; St. Louis, MO) and propranolol (Cat. No. P8688, Sigma-Aldrich; St. Louis, MO).

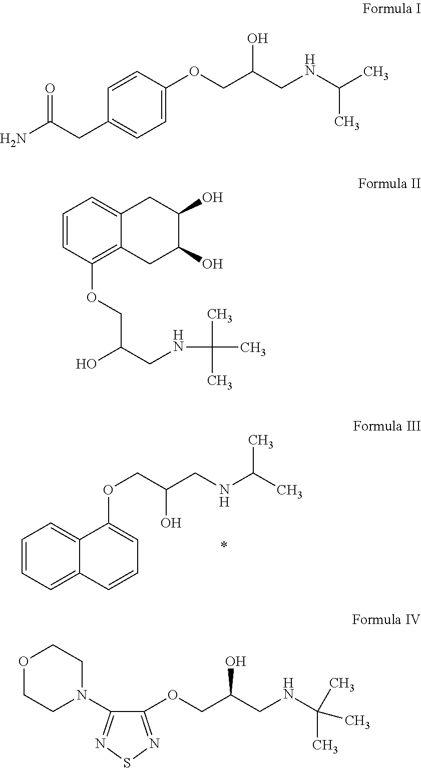 [SMILES: CC(C)NCC(O)COc1ccc(CC(N)=O)cc1 ; CC(C)(C)NCC(O)COc1cccc2c1C[C@H](O)[C@H](O)C2 ; CC(C)NCC(O)COc1cccc2ccccc12 ; CC(C)(C)NC[C@H](O)COc1nsnc1N1CCOCC1]

In some embodiments of any of the aspects, the at least one beta-blocker is selected from the group consisting of atenolol; R+ atenolol; nadolol; propranolol; R+ propranolol; and timolol.

In some embodiments of any of the aspects, the at least one beta-blocker comprises atenolol. In some embodiments of any of the aspects, the at least one beta-blocker comprises R+ atenolol. In some embodiments of any of the aspects, the at least one beta-blocker comprises nadolol. In some embodiments of any of the aspects, the at least one beta-blocker comprises propranolol. In some embodiments of any of the aspects, the at least one beta-blocker comprises R+ propranolol. In some embodiments of any of the aspects, the at least one beta-blocker comprises timolol.

In some embodiments of any of the aspects, the at least one beta-blocker consists of atenolol. In some embodiments of any of the aspects, the at least one beta-blocker consists of R+ atenolol. In some embodiments of any of the aspects, the at least one beta-blocker consists of nadolol. In some embodiments of any of the aspects, the at least one beta-blocker consists of propranolol. In some embodiments of any of the aspects, the at least one beta-blocker consists of R+ propranolol. In some embodiments of any of the aspects, the at least one beta-blocker consists of timolol.

In some embodiments of any of the aspects, the subject is administered simvastatin and atenolol. In some embodiments of any of the aspects, the subject is administered simvastatin and R+ atenolol. In some embodiments of any of the aspects, the subject is administered simvastatin and nadolol. In some embodiments of any of the aspects, the subject is administered simvastatin and propranolol. In some embodiments of any of the aspects, the subject is administered simvastatin and R+ propranolol. In some embodiments of any of the aspects, the subject is administered simvastatin and timolol.

In some embodiments of any of the aspects, the subject is administered atorvastatin and atenolol. In some embodiments of any of the aspects, the subject is administered atorvastatin and R+ atenolol. In some embodiments of any of the aspects, the subject is administered atorvastatin and nadolol. In some embodiments of any of the aspects, the subject is administered atorvastatin and propranolol. In some embodiments of any of the aspects, the subject is administered atorvastatin and R+ propranolol. In some embodiments of any of the aspects, the subject is administered atorvastatin and timolol.

The mTOR inhibitor rapamycin (sirolimus) reduces stem cell properties of hemangioma stem cells and blocks vessel formation in vivo. It was shown that a 4 day treatment with rapamycin in vitro, prior to implantation in vivo, blocked hemangioma vessel formation (Greenberger et al. J Invest Dermatol 2011 131:2467-76 (PMID: 21938011); which is incorporated by reference herein in its entirety. That is, mTOR inhibitors exert a distinct inhibitory effect on hemangioma stem cells, and it is contemplated herein that this therapeutic effect can be combined, additively or synergistically, with the therapeutic effect of statins demonstrated herein.

As used herein, the term “mTOR inhibitor” refers to an agent that can reduce the expression level and/or activity of mTOR protein and/or mRNA. In some embodiments, an mTOR inhibitor can reduce the expression level of mTOR mRNA. In some embodiments, an mTOR inhibitor can reduce the expression level of mTOR polypeptide. In some embodiments, an mTOR inhibitor can reduce the activity of mTOR polypeptide. As used herein, the term “mTOR” refers to a serine/threonine kinase of the PI3K enzyme family that functions as the catalytic subunit of the mTORC1 and mTORC2 complexes (e.g. NCBI Gene ID: 2475). mTOR is also referred to as FRAP, RAFT1, and RAPT. mTOR inhibitors can inhibit mTOR via any known mechanism, including, e.g., binding of a competitive inhibitor, binding of a non-competitive inhibitor, increasing the rate of degradation of mTOR polypeptides, blocking the biosynthesis, transcription, and/or translation of mTOR, blocking the targeting of AKT to mTOR, and increasing the inhibition of mTOR by TSC1/2. mTOR inhibition can be determined by methods well known in the art, e.g. by detecting the level of phosphorylation of mTOR targets such as p70-S6 kinase 1 (S6K1), 4E-BP1, and Akt, where inhibition or a decreased in the phosphorylation of these targets indicates effective inhibition of mTOR. In certain embodiments, an agent can increase or decrease the expression of a component of the targeted signaling pathway. Components of the mTOR signaling pathway include, but are not limited to RAPTOR, DEPTOR, Rheb, AKT, RICTOR, GβL, and HIF-1. The mTOR signaling pathways have been described in the art, e.g. in Dunlop and Tee Cell Signal 2009 26:827-835; Laplante and Sabatini J Cell Sci 2009 122:3589-3594; Kudchodkar et al. PNAS 2006 103:14182-7; which are incorporated by reference herein in their entireties. Transcriptional assays are well known to those of skill in the art (see e.g. U.S. Pat. Nos. 7,319,933, 6,913,880 which is incorporated herein by reference in its entirety).

Non-limiting examples of mTOR inhibitors for use in the methods and compositions described herein include everolimus (e.g. a compound having the structure of Formula V), temsirolimus (e.g. a compound having the structure of Formula VI), sirolimus (also referred to in the art as rapamycin) (e.g. a compound having the structure of Formula VII), deforolimus, TOP216, OSI-027, TAFA93, nab-rapamycin, tacrolimus, biolimus, CI-779, ABT-578, AP-23675, BEZ-235, QLT-0447, ABI-009, BC-210, salirasib, AP-23841, AP-23573, KU-0059475, 32-deoxorapamycin, 16-pent-2-ynyloxy-32-deoxorapamycin, 16-pent-2-ynyloxy-32 (S or R)-dihydro-rapamycin, 16-pent-2-ynyloxy-32 (S or R)-dihydro-40-O-(2-hydroxyethyl)-rapamycin, 40-O-(2-hydroxyethyl)-rapamycin, 32-deoxorapamycin; 16-pent-2-ynyloxy-32 (S)-dihydrorapamycin; socalledrapalogs; AP23464; PI-103, PP242, PP30, Torin1; and derivatives or pharmaceutically acceptable salts thereof as well as and compounds described in, e.g. U.S. Patent Publications 2011/0178070; 2011/0021515; 2007/0112005; 2011/0054013; International Patent Publications WO98/02441; WO01/14387; WO99/15530; WO07/135411; WO03/64383; WO96/41807; WO95/16691; WO94/09010; European Patent No. EP1880723; and U.S. Pat. Nos. 8,163,775; 6,329,386; 6,200,985; 6,117,863; 6,015,815; 6,015,809; 6,004,973; 5,985,890; 5,955,457; 5,922,730; 5,912,253; 5,780,462; 5,665,772, 5,637,590; 5,567,709; 5,563,145; 5,559, 122; 5,559, 120; 5,559,119; 5,559, 112; 5,550, 133; 5,541, 192; 5,541, 191; 5,532,355; 5,530, 121; 5,530,007; 5,525,610; 5,521,194; 5,519,031; 5,516,780; 5,508,399; 5,508,290; 5,508,286; 5,508,285; 5,504,291; 5,504,204; 5,491,231; 5,489,680; 5,489,595; 5,488,054; 5,486,524; 5,486,523; 5,486,522; 5,484,791; 5,484,790; 5,480,989; 5,480,988; 5,463,048; 5,446,048; 5,434,260; 5,411,967; 5,391,730; 5,389,639; 5,385,910; 5,385,909; 5,385,908; 5,378,836; 5,378,696; 5,373,014; 5,362,718; 5,358,944; 5,346,893; 5,344,833; 5,302,584; 5,262,424; 5,262,423; 5,260,300; 5,260,299; 5,233,036; 5,221,740; 5,221,670; 5,202,332; 5,194,447; 5,177,203; 5,169,851; 5,164,399; 5, 162,333; 5,151,413; 5,138,051; 5,130,307; 5,120,842; 5,120,727; 5,120,726, 5,120,725; 5,118,678; 5,118,677; 5,100,883; 5,023,264; 5,023,263; and 5,023,262; which are incorporated by reference herein in their entireties.

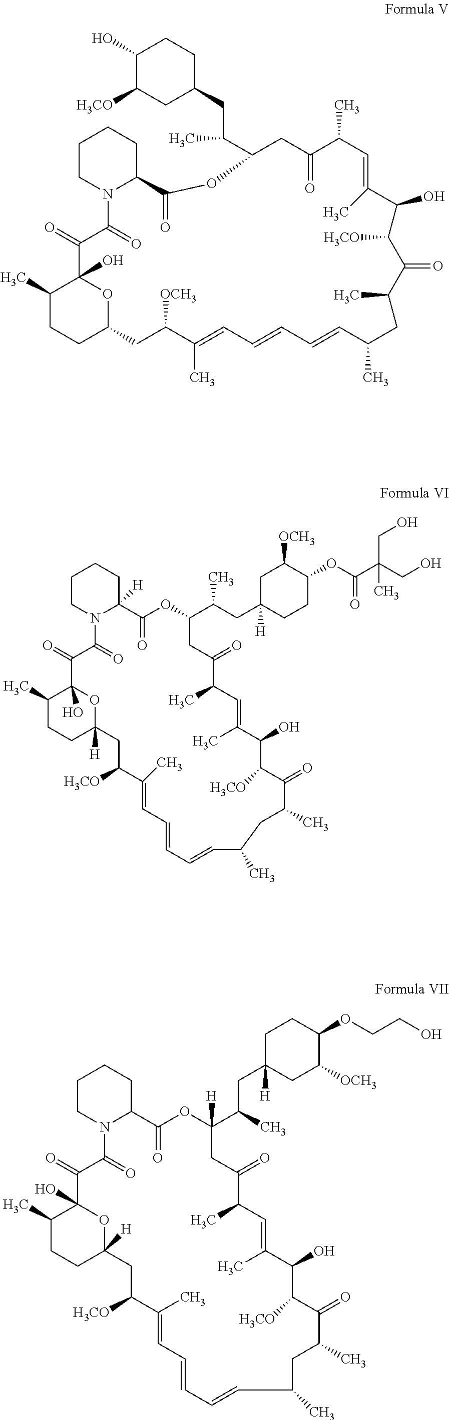

In some embodiments of any of the aspects, the at least one mTOR inhibitor is selected from the group consisting of sirolumis; everolimus; temisirolimus; and rapamycin.

In some embodiments of any of the aspects, the at least one mTOR inhibitor comprises sirolumis. In some embodiments of any of the aspects, the at least one mTOR inhibitor comprises everolimus. In some embodiments of any of the aspects, the at least one mTOR inhibitor comprises temisirolimus. In some embodiments of any of the aspects, the at least one mTOR inhibitor comprises rapamycin.

In some embodiments of any of the aspects, the at least one mTOR inhibitor consists of sirolumis. In some embodiments of any of the aspects, the at least one mTOR inhibitor consists of everolimus. In some embodiments of any of the aspects, the at least one mTOR inhibitor consists of temisirolimus. In some embodiments of any of the aspects, the at least one mTOR inhibitor consists of rapamycin.

In some embodiments of any of the aspects, the subject is administered simvastatin and sirolimus. In some embodiments of any of the aspects, the subject is administered simvastatin and everolimus. In some embodiments of any of the aspects, the subject is administered simvastatin and temsirolimus. In some embodiments of any of the aspects, the subject is administered simvastatin and rapamycin.

In some embodiments of any of the aspects, the subject is administered atorvastatin and sirolimus. In some embodiments of any of the aspects, the subject is administered atorvastatin and everolimus. In some embodiments of any of the aspects, the subject is administered atorvastatin and temsirolimus. In some embodiments of any of the aspects, the subject is administered atorvastatin and rapamycin.

In some embodiments, the steroid can be a corticosteroid. As used herein, the term “steroid” refers to a chemical substance comprising three cyclohexane rings and a cyclopentane ring. The rings are arranged to form tetracyclic cyclopentaphenanthrene, i.e. gonane. As used herein, the term “corticosteroid” refers to a class of steroid hormones that are produced in the adrenal cortex or produced synthetically. Corticosteroids are involved in a wide range of physiologic systems such as stress response, immune response and regulation of inflammation, carbohydrate metabolism, protein catabolism, blood electrolyte levels, and behavior. Corticosteroids are generally grouped into four classes, based on chemical structure. Group A corticosteroids (short to medium acting glucocorticoids) include hydrocortisone, hydrocortisone acetate, cortisone acetate, tixocortol pivalate, prednisolone, methylprednisolone, and prednisone. Group B corticosteroids include triamcinolone acetonide, triamcinolone alcohol, mometasone, amcinonide, budesonide, desonide, fluocinonide, fluocinolone acetonide, and halcinonide. Group C corticosteroids include betamethasone, betamethasone sodium phosphate, dexamethasone, dexamethasone sodium phosphate, and fluocortolone. Group D corticosteroids include hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, and fluprednidene acetate. Non-limiting examples of corticosteroids include, aldosternone, beclomethasone, beclomethasone dipropionate, betametahasone, betametahasone-21-phosphate disodium, betametahasone valerate, budesonide, clobetasol, clobetasol propionate, clobetasone butyrate, clocortolone pivalate, cortisol, cortisteron, cortisone, deflazacort, dexamethasone, dexamethasone acetate, dexamethasone sodium phosphate, diflorasone diacetate, dihydroxycortison, flucinonide, fludrocortisones acetate, flumethasone, flunisolide, flucionolone acetonide, fluticasone furate, fluticasone propionate, halcinonide, halpmetasone, hydrocortisone, hydroconrtisone acetate, hydrocortisone succinate, 16α-hydroxyprednisolone, isoflupredone acetate, medrysone, methylprednisolone, prednacinolone, predricarbate, prednisolone, prednisolone acetate, prednisolone sodium succinate, prednisone, triamcinolone, triamcinolone, and triamcinolone diacetate. As used herein, the term “corticosteroid” can include, but is not limited to, the following generic and brand name corticosteroids: cortisone (CORTONE™ ACETATE™, ADRESON™, ALTESONA™, CORTELAN™, CORTISTAB™, CORTISYL™, CORTOGEN™, CORTONE™, SCHEROSON™); dexamethasone-oral (DECADRON-ORAL™, DEXAMETH™, DEXONE™, HEXADROL-ORAL™, DEXAMETHASONE™ INTENSOL™, DEXONE 0.5™, DEXONE 0.75™, DEXONE 1.5™, DEXONE 4™); hydrocortisone-oral (CORTEF™, HYDROCORTONE™); hydrocortisone cypionate (CORTEF ORAL SUSPENSION™); methylprednisolone-oral (MEDROL-ORAL™); prednisolone-oral (PRELONE™, DELTA-CORTEF™, PEDIAPRED™, ADNISOLONE™, CORTALONE™, DELTACORTRIL™, DELTASOLONE™, DELTASTAB™, DI-ADRESON F™, ENCORTOLONE™, HYDROCORTANCYL™, MEDISOLONE™, METICORTELONE™, OPREDSONE™, PANAAFCORTELONE™, PRECORTISYL™, PRENISOLONA™, SCHERISOLONA™, SCHERISOLONE™); prednisone (DELTASONE™, LIQUID PRED™, METICORTEN™, ORASONE 1™, ORASONE 5™, ORASONE 10™, ORASONE 20™, ORASONE 50™, PREDNICEN-M™, PREDNISONE INTENSOL™, STERAPRED™, STERAPRED DS™, ADASONE™, CARTANCYL™, COLISONE™, CORDROL™, CORTAN™, DACORTIN™, DECORTIN™, DECORTISYL™, DELCORTIN™, DELLACORT™, DELTADOME™, DELTACORTENE™, DELTISONA™, DIADRESON™, ECONOSONE™, ENCORTON™, FERNISONE™, NISONA™, NOVOPREDNISONE™ PANAFCORT™, PANASOL™, PARACORT™, PARMENISON™, PEHACORT™, PREDELTIN™, PREDNICORT™, PREDNICOT™, PREDNIDIB™, PREDNIMENT™, RECTODELT™, ULTRACORTEN™, WINPRED™); triamcinoloneoral (KENACORT™, ARISTOCORT™, ATOLONE™, SHOLOG ATM, TRAMACORT-D™, TRI-MED™ TRIAMCOT™, TRISTOPLEX™, TRYLONE D™, U-TRI-LONE™). In some embodiments, a corticosteroid can be a corticosteroid which is active when applied topically, including, but not limited to clobetasol propionate, betamethasone valerate, betamethasone dripropionate, and mometasone furoate. In some embodiments, a corticosteroid can be dexamethasone (e.g. a compound having the structure of Formula VIII); prednisone (e.g. a compound having the structure of Formula IX); prednisolone (e.g. a compound having the structure of Formula X); triamcinolone (e.g. a compound having the structure of Formula XI); clobetasol propionate (e.g. a compound having the structure of Formula XII); betamethasone valerate (e.g. a compound having the structure of Formula XIII); betamethasone dipropionate (e.g. a compound having the structure of Formula XIV); or mometasone furoate (e.g. a compound having the structure of Formula XV). Methods of synthesizing steroids and corticosteroids are well known in the art and such compounds are also commercially available, e.g. dexamethasone (Cat. No. D4902, Sigma-Aldrich; St. Louis, MO) and predinsone (Cat. No. P6254, Sigma-Aldrich; St. Louis, MO).

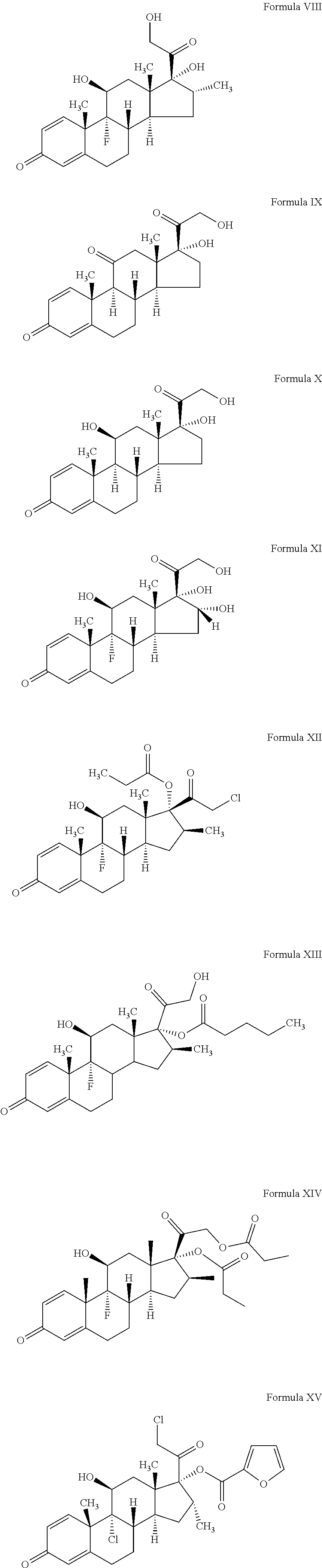

In some embodiments of any of the aspects, the at least one corticosteroid is selected from the group consisting of dexamethasone; prednisone; prednisolone; triamcinolone; clobetasol propionate; betamethasone valerate; betamethasone dipropionate; and mometasone furoate.

In some embodiments of any of the aspects, the at least one corticosteroid comprises dexamethasone. In some embodiments of any of the aspects, the at least one corticosteroid comprises prednisone. In some embodiments of any of the aspects, the at least one corticosteroid comprises prednisolone. In some embodiments of any of the aspects, the at least one corticosteroid comprises triamcinolone. In some embodiments of any of the aspects, the at least one corticosteroid comprises clobetasol propionate. In some embodiments of any of the aspects, the at least one corticosteroid comprises betamethasone valerate. In some embodiments of any of the aspects, the at least one corticosteroid comprises betamethasone dipropionate. In some embodiments of any of the aspects, the at least one corticosteroid comprises mometasone furoate.

In some embodiments of any of the aspects, the at least one corticosteroid consists of dexamethasone. In some embodiments of any of the aspects, the at least one corticosteroid consists of prednisone. In some embodiments of any of the aspects, the at least one corticosteroid consists of prednisolone. In some embodiments of any of the aspects, the at least one corticosteroid consists of triamcinolone. In some embodiments of any of the aspects, the at least one corticosteroid consists of clobetasol propionate. In some embodiments of any of the aspects, the at least one corticosteroid consists of betamethasone valerate. In some embodiments of any of the aspects, the at least one corticosteroid consists of betamethasone dipropionate. In some embodiments of any of the aspects, the at least one corticosteroid consists of mometasone furoate.

In some embodiments of any of the aspects, the subject is administered simvastatin and dexamethasone. In some embodiments of any of the aspects, the subject is administered simvastatin and prednisone. In some embodiments of any of the aspects, the subject is administered simvastatin and prednisolone. In some embodiments of any of the aspects, the subject is administered simvastatin and triamcinolone. In some embodiments of any of the aspects, the subject is administered simvastatin and clobetasol propionate. In some embodiments of any of the aspects, the subject is administered simvastatin and betamethasone valerate. In some embodiments of any of the aspects, the subject is administered simvastatin and betamethasone dipropionate. In some embodiments of any of the aspects, the subject is administered simvastatin and mometasone furoate.

In some embodiments of any of the aspects, the subject is administered atorvastatin and dexamethasone. In some embodiments of any of the aspects, the subject is administered atorvastatin and prednisone. In some embodiments of any of the aspects, the subject is administered atorvastatin and prednisolone. In some embodiments of any of the aspects, the subject is administered atorvastatin and triamcinolone. In some embodiments of any of the aspects, the subject is administered atorvastatin and clobetasol propionate. In some embodiments of any of the aspects, the subject is administered atorvastatin and betamethasone valerate. In some embodiments of any of the aspects, the subject is administered atorvastatin and betamethasone dipropionate. In some embodiments of any of the aspects, the subject is administered atorvastatin and mometasone furoate.

In some embodiments of any of the aspects, the subject is further administered nifedipine (e.g. a compound having the structure of Formula XVI); antimycin A; chelidonine monohydrate; lycorine hydrochloride; ionomycin; LY-294,002; cerulenin; or monensin sodium.

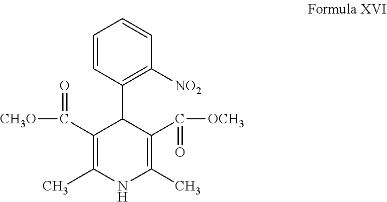

In some embodiments of any of the aspects, the subject is further administered antimycin A. In some embodiments of any of the aspects, the subject is further administered chelidonine monohydrate. In some embodiments of any of the aspects, the subject is further administered lycorine hydrochloride. In some embodiments of any of the aspects, the subject is further administered ionomycin. In some embodiments of any of the aspects, the subject is further administered LY-294,002. In some embodiments of any of the aspects, the subject is further administered cerulenin. In some embodiments of any of the aspects, the subject is further administered monensin sodium.

In some embodiments, the methods described herein relate to administering to a subject having, or diagnosed as having, a vascular anomaly at least one statin. Subjects having a vascular anomaly can be identified by a physician using current methods of diagnosing vascular anomalies, as described herein. Symptoms of vascular anomalies which characterize these conditions and aid in diagnosis are well known in the art and include but are not limited to, discolorations of the epidermis or the presence of a vascular tissue tumor. Tests that aid in a diagnosis of vascular anomalies, e.g. hemangioma, include, but are not limited to, ultrasound, MRI, and histochemical examination of biopsies. A family history of hemangiomas can also aid in determining if a subject is likely to have a hemangioma or in making a diagnosis of hemangioma.

The compositions and methods described herein can be administered to a subject having, or diagnosed as having, a vascular anomaly. In some embodiments, the methods described herein comprise administering an effective amount of a composition described herein, e.g. a statin, to a subject in order to alleviate a symptom of a vascular anomaly. As used herein, “alleviating a symptom” is ameliorating any condition or symptom associated with the vascular anomaly, e.g. tumor size, extent of the irregular vasculature, or growth of irregular vasculature. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, intralesionally, or intratumoral administration. Administration can be local or systemic.

In some embodiments, the at least one statin is administered orally. In some embodiments, the at least one statin is administered orally in the form of a syrup or suspension, optionally with an added flavoring.

In some embodiments, an mTOR inhibitor, steroid, and/or beta-blocker can be administered topically. In some embodiments, an mTOR inhibitor, steroid, and/or beta-blocker can be administered orally.

The term “effective amount” as used herein refers to the amount of composition needed to alleviate at least one or more symptoms of the disease or disorder, and relates to a sufficient amount of pharmacological composition to provide the desired effect. The term “therapeutically effective amount” therefore refers to an amount of a composition that is sufficient to effect a particular anti-vasculogenesis effect when administered to a typical subject. An effective amount as used herein in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays as described herein. Also, a dose can be formulated in animal models to achieve a circulating plasma concentration range that includes the IC50 (i.e., the concentration of the active ingredient which achieves a half-maximal inhibition of a symptom) as determined in cell culture, or in an appropriate animal model. The level of a statin in plasma can be measured, for example, by high performance liquid chromatography. The effects of any particular dosage can be monitored by a suitable bioassay, e.g., an in vitro or in vivo assay for vasculogenesis or tumor size among others. The dosage can be determined by a physician and adjusted, as necessary, to suit observed effects of the treatment.

In some embodiments, the technology described herein relates to a pharmaceutical composition comprising at least one statin, and optionally, a steroid, mTOR inhibitor and/or beta-blocker as described herein, and optionally a pharmaceutically acceptable carrier. In some embodiments, the composition can comprise multiple statins, multiple steroids, multiple mTOR inhibitors, multiple beta-blockers, or any combination thereof.

In one aspect of any of the embodiments, described herein is a combination or kit comprising at least one statin and at least one beta-blocker, mTOR inhibitor, and/or steroid. In some embodiments, the a) at least one statin and b) at least one beta-blocker, mTOR inhibitor, and/or steroid are provided in the same formulation. In some embodiments, the a) at least one statin and b) at least one beta-blocker, mTOR inhibitor, and/or steroid are provided in separate formulations.

As used herein “combination” refers to a group of two or more substances for use together, e.g., for use in treating vascular anomalies. The two or more substances can be present in the same formulation in any molecular or physical arrangement, e.g., in an admixture, in a solution, in a mixture, in a suspension, in a colloid, in an emulsion. The formulation can be a homogeneous or heterogenous mixture. In some embodiments of any of the aspects, the two or more substances active compound(s) can be comprised by the same or different superstructures, e.g., nanoparticles, liposomes, vectors, cells, scaffolds, or the like, and said superstructure is in solution, mixture, admixture, suspension with a solvent, carrier, or some of the two or more substances. Alternatively, the two or more substances can be present in two or more separate formulations, e.g., in a kit or package comprising multiple formulations in separate containers, to be mixed or brought into contact with each other when an assay is to be performed.

A kit is an assemblage of materials or components, including at least one reagent described herein. The exact nature of the components configured in the kit depends on its intended purpose. In some embodiments of any of the aspects, a kit includes instructions for use. “Instructions for use” typically include a tangible expression describing the technique to be employed in using the components of the kit, e.g., to treat a vascular anomaly. Still in accordance with the present invention, “instructions for use” may include a tangible expression describing the preparation of at least one element described herein, such as dilution, mixing, or dosing instructions, and the like, typically for an intended purpose. Optionally, the kit also contains other useful components, such as, measuring tools, diluents, buffers, syringes, pharmaceutically acceptable carriers, or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging may also preferably provide an environment that protects from light, humidity, and oxygen. As used herein, the term “package” refers to a suitable solid matrix or material such as glass, plastic, paper, foil, polyester (such as polyethylene terephthalate, or Mylar) and the like, capable of holding the individual kit components. Thus, for example, a package can be a glass vial used to contain suitable quantities of a composition containing a volume of at least one reagent described herein. The packaging material generally has an external label which indicates the contents and/or purpose of the kit and/or its components.

In some embodiments of any of the aspects, the kit or combination comprises at least one statin and at least one beta-blocker. In some embodiments of any of the aspects, the kit or combination comprises at least one statin and at least one mTOR inhibitor. In some embodiments of any of the aspects, the kit or combination comprises at least one statin and at least one steroid.

In some embodiments of any of the aspects, the combination or kit comprises simvastatin and atenolol. In some embodiments of any of the aspects, the combination or kit comprises simvastatin and nadolol. In some embodiments of any of the aspects, the combination or kit comprises simvastatin and propranolol. In some embodiments of any of the aspects, the combination or kit comprises simvastatin and R+ propranolol. In some embodiments of any of the aspects, the combination or kit comprises simvastatin and timolol.

In some embodiments of any of the aspects, the combination or kit comprises atorvastatin and atenolol. In some embodiments of any of the aspects, the combination or kit comprises atorvastatin and nadolol. In some embodiments of any of the aspects, the combination or kit comprises atorvastatin and propranolol. In some embodiments of any of the aspects, the combination or kit comprises atorvastatin and R+ propranolol. In some embodiments of any of the aspects, the combination or kit comprises atorvastatin and timolol.

In some embodiments of any of the aspects, the combination or kit comprises simvastatin and sirolimus. In some embodiments of any of the aspects, the combination or kit comprises simvastatin and everolimus. In some embodiments of any of the aspects, the combination or kit comprises simvastatin and temsirolimus. In some embodiments of any of the aspects, the combination or kit comprises simvastatin and rapamycin.

In some embodiments of any of the aspects, the combination or kit comprises atorvastatin and sirolimus. In some embodiments of any of the aspects, the combination or kit comprises atorvastatin and everolimus. In some embodiments of any of the aspects, the combination or kit comprises atorvastatin and temsirolimus. In some embodiments of any of the aspects, the combination or kit comprises atorvastatin and rapamycin.

In some embodiments of any of the aspects, the combination or kit comprises simvastatin and dexamethasone. In some embodiments of any of the aspects, the combination or kit comprises simvastatin and prednisone. In some embodiments of any of the aspects, the combination or kit comprises simvastatin and prednisolone. In some embodiments of any of the aspects, the combination or kit comprises simvastatin and triamcinolone. In some embodiments of any of the aspects, the combination or kit comprises simvastatin and clobetasol propionate. In some embodiments of any of the aspects, the combination or kit comprises simvastatin and betamethasone valerate. In some embodiments of any of the aspects, the combination or kit comprises simvastatin and betamethasone dipropionate. In some embodiments of any of the aspects, the combination or kit comprises simvastatin and mometasone furoate.

In some embodiments of any of the aspects, the combination or kit comprises atorvastatin and dexamethasone. In some embodiments of any of the aspects, the combination or kit comprises atorvastatin and prednisone. In some embodiments of any of the aspects, the combination or kit comprises atorvastatin and prednisolone. In some embodiments of any of the aspects, the combination or kit comprises atorvastatin and triamcinolone. In some embodiments of any of the aspects, the combination or kit comprises atorvastatin and clobetasol propionate. In some embodiments of any of the aspects, the combination or kit comprises atorvastatin and betamethasone valerate. In some embodiments of any of the aspects, the combination or kit comprises atorvastatin and betamethasone dipropionate. In some embodiments of any of the aspects, the combination or kit comprises atorvastatin and mometasone furoate.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent as described herein.

In some embodiments a composition described herein can be administered to a subject topically. In some embodiments, topical dosage forms can include, but are not limited to, creams, lotions, ointments, gels, shampoos, sprays, aerosols, solutions, emulsions, patches, and other forms known to one of skill in the art. See, e.g., Remington: The Science and Practice of Pharmacy, 21st Ed., Lippincott, Williams, and Wilkins, Philadelphia PA. (2005); and Ansel's Pharmaceutical Dosage Forms and Drug Delivery Systems, 9th Ed., Lippincott, Williams, and Wilkins, Philadelphia, PA. (2011). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, e.g. a statin, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. See, e.g., Remington: The Science and Practice of Pharmacy, 21st Ed., Lippincott, Williams, and Wilkins, Philadelphia PA. (2005).

Transdermal and mucosal dosage forms of the compositions described herein but are not limited to, patches, sprays, aerosols, creams, lotions, suppositories, ointments, gels, solutions, emulsions, suspensions, or other forms known to one of skill in the art. See, e.g., Remington: The Science and Practice of Pharmacy, 21st Ed., Lippincott, Williams, and Wilkins, Philadelphia PA. (2005); and Ansel's Pharmaceutical Dosage Forms and Drug Delivery Systems, 9th Ed., Lippincott, Williams, and Wilkins, Philadelphia, PA. (2011). Dosage forms suitable for treating mucosal tissues within the oral cavity can be formulated as mouthwashes, as oral gels, or as buccal patches. Additional transdermal dosage forms include “reservoir type” or “matrix type” patches, which can be applied to the skin and worn for a specific period of time to permit the penetration of a desired amount of active ingredient.

Suitable excipients (e.g., carriers and diluents) and other materials that can be used to provide transdermal and mucosal dosage forms encompassed by this disclosure are well known to those skilled in the pharmaceutical arts, and depend on the particular tissue or organ to which a given pharmaceutical composition or dosage form will be applied. With that fact in mind, typical excipients include, but are not limited to water, acetone, ethanol, ethylene glycol, propylene glycol, butane-1,3-diol, isopropyl myristate, isopropyl palmitate, mineral oil, and mixtures thereof, to form dosage forms that are non-toxic and pharmaceutically acceptable. In some embodiments, penetration enhancers can be used to assist in delivering the active ingredients to or across the tissue. Suitable penetration enhancers include, but are not limited to: acetone; various alcohols such as ethanol, oleyl, an tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as TWEEN 80 (polysorbate 80) and SPAN 60 (sorbitan monostearate).

In some embodiments, the pharmaceutical composition described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, administration DUROS®-type dosage forms, and dose-dumping.

Suitable vehicles that can be used to provide parenteral dosage forms are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of an agent as disclosed herein can also be incorporated into the parenteral dosage forms of the disclosure, including conventional and controlled-release parenteral dosage forms.

Pharmaceutical compositions described herein can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia PA. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, composition described herein can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

The methods described herein can further comprise administering a second agent and/or further treatment to the subject, e.g. as part of a combinatorial therapy. Non-limiting examples of a second agent and/or further treatment can include one or more of laser surgery, pulsed dye laser, corticosteroid therapy, oral corticosteroid therapy, beta-blockers, topical beta-blockers, vincristine, interferon, surgical removal, and pharmaceutically acceptable salts, acids or derivatives of any of the preceding compounds.

In certain embodiments, an effective dose of a composition as described herein can be administered to a patient once. In certain embodiments, an effective dose of a composition described herein can be administered to a patient repeatedly. For systemic administration, subjects can be administered a therapeutic amount of at least one statin, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more. A composition comprising at least one statin can be administered over a period of time, such as over a 5 minute, 10 minute, 15 minute, 20 minute, or 25 minute period or longer. The administration can be repeated, for example, on a regular basis, such as hourly, every 3 hours, 6 hours, 12 hours or longer or such as biweekly (i.e., every two weeks) for one month, two months, three months, four months or longer.

In some embodiments, after an initial treatment regimen, the at least one statin can be administered on a less frequent basis. As a non-limiting example, after treatment biweekly for three months, treatment can be repeated once per month, for six months or a year or longer. Treatment according to the methods described herein can reduce levels of a marker or symptom of a condition, e.g. proliferative vascular disorder by at least 10%, at least 15%, at least 20%, at least 25%, at least 30%, at least 40%, at least 50%, at least 60%, at least 70%, at least 80% or at least 90% or more. Markers and symptoms of proliferative vascular disorders, e.g. hemangioma can include, but are not limited to tumors of the endothelial cells of blood vessels; an increased number of blood vessels and/or capillaries; discoloration of the skin; GLUT-1 expression; hemosiderin pigmentation; ulceration; and bleeding.

When administered at different times, a composition comprising at least one statin and a composition comprising a further agent as described herein can be administered substantially simultaneously with each other or within 5 minutes, or 10 minutes, or 20 minutes, or 60 minutes, or 2 hours, or 3 hours, or 4 hours, or 8 hours, or 12 hours, or 24 hours of administration of the other. When at least one statin and at least one further agent as described herein are administered in different pharmaceutical compositions, routes of administration can be the same or different. For example, a composition comprising a statin can be administered by any appropriate route known in the art including, but not limited to orally or systemically, and a beta-blocker can be administered by a different route, e.g. topically, or a route commonly used in the art for administration of the beta-blocker.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to a statin and any further agents administered. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more.

The dosage ranges for the administration of a statin, and optionally, a further agent described herein, according to the methods described herein depend upon, for example, the form of the compound(s), its potency, and the extent to which symptoms, markers, or indicators of a condition described herein are desired to be reduced, for example the percentage reduction desired for vasculogenesis and/or tumor size. The dosage should not be so large as to cause adverse side effects, such as thrombocytopenia. Generally, the dosage will vary with the age, condition, and sex of the patient and can be determined by one of skill in the art. The dosage can also be adjusted by the individual physician in the event of any complication.

The efficacy of a statin, e.g., the treatment of a condition described herein, or to induce a response as described herein (e.g., reduction of tumor size, extent, or rate of growth) can be determined by the skilled clinician. However, a treatment is considered “effective treatment,” as the term is used herein, if any one or all of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., extent of vasculogenesis and/or tumor size. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g., decrease in tumor size). It is well within the ability of one skilled in the art to monitor efficacy of administration and/or treatment by measuring any one of such parameters, or any combination of parameters. When using an experimental animal model, efficacy of treatment is evidenced when a statistically significant change in a marker is observed, e.g. extent of vascularization and/or tumor size.

In vitro and animal model assays are provided herein which allow the assessment of a given dose of a statin. By way of non-limiting example, the effects of a dose of a statin proliferation is inhibited by the compound in an in vitro or in vivo assay described herein or known in the art as a model for a vascular anomaly.

In one respect, the present invention relates to the herein described compositions, methods, and respective component(s) thereof, as essential to the technology, yet open to the inclusion of unspecified elements, essential or not (“comprising). In some embodiments of any of the aspects, other elements to be included in the description of the composition, method or respective component thereof are limited to those that do not materially affect the basic and novel characteristic(s) of the technology (e.g., the composition, method, or respective component thereof “consists essentially of” the elements described herein). This applies equally to steps within a described method as well as compositions and components therein. In other embodiments of any of the aspects, the compositions, methods, and respective components thereof, described herein are intended to be exclusive of any element not deemed an essential element to the component, composition or method (e.g., the composition, method, or respective component thereof “consists of” the elements described herein). This applies equally to steps within a described method as well as compositions and components therein.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomolgous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a vascular anomaly. A subject can be male or female.

A subject can be one who has been previously diagnosed with or identified as suffering from or having a condition in need of treatment (e.g. vascular anomaly) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a vascular anomaly. The term “treating” includes reducing or alleviating at least one adverse effect or symptom of a condition, disease or disorder associated with a vascular anomaly. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

In some embodiments, treatment is effective if the rate of growth of a vascular anomaly is decreased. In some embodiments, treatment is effective if the size of a vascular anomaly is decreased. In some embodiments, treatment is effective if the visible appearance of a vascular anomaly is decreased.

In some embodiments of any of the aspects, described herein is a prophylactic method of treatment. As used herein “prophylactic” refers to the timing and intent of a treatment relative to a disease or symptom, that is, the treatment is administered prior to clinical detection or diagnosis of that particular disease or symptom in order to protect the patient from the disease or symptom. Prophylactic treatment can encompass a reduction in the severity or speed of onset of the disease or symptom, or contribute to faster recovery from the disease or symptom. Accordingly, the methods described herein can be prophylactic relative to further growth of a vascular anomaly. In some embodiments of any of the aspects, prophylactic treatment is not prevention of all symptoms or signs of a disease.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “nanoparticle” refers to particles that are on the order of about 1 to 1,000 nanometers in diameter or width. The term “nanoparticle” includes nanospheres; nanorods; nanoshells; and nanoprisms; these nanoparticles may be part of a nanonetwork. The term “nanoparticles” also encompasses liposomes and lipid particles having the size of a nanoparticle. Exemplary nanoparticles include lipid nanoparticles or ferritin nanoparticles. Lipid nanoparticles can comprise multiple components, including, e.g., ionizable lipids (such as MC3, DLin-MC3-DMA, ALC-0315, or SM-102), pegylated lipids (such as PEG2000-C-DMG, PEG2000-DMG, ALC-0159), phospholipids (such as DSPC), and cholesterol.

Exemplary liposomes can comprise, e.g., DSPC, DPPC, DSPG, Cholesterol, hydrogenated soy phosphatidylcholine, soy phosphatidyl choline, methoxypolyethylene glycol (mPEG-DSPE) phosphatidyl choline (PC), phosphatidyl glycerol (PG), distearoylphosphatidylcholine, and combinations thereof.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

In some embodiments, the present technology may be defined in any of the following numbered paragraphs:
1. A method of treating a vascular anomaly in a subject in need thereof, the method comprising administering a composition comprising at least one statin to the subject.
2. The method of any one of the preceding paragraphs, wherein the statin is selected from the group consisting of:
   simvastatin; atorvastatin; cerivastatin; fluvastatin; lovastatin; mevastatin; pitavastatin;
   pravastatin; and rosuvastatin.
3. The method of any one of the preceding paragraphs, wherein the statin is simvastatin.
4. The method of any one of the preceding paragraphs, wherein the statin is atorvastatin.
5. The method of any one of the preceding paragraphs, wherein the vascular anomaly is selected from the group consisting of:
   infantile hemangioma, congenital hemangioma, pyogenic granuloma, tufted angioma, kaposiform hemangioendothelioma, epitheloid hemangioendothelioma (EHE), capillary malformations, arteriovenous malformations, venous malformations, lymphatic malformations, complex lymphatic malformations, generalized lymphatic anomaly, central conducting lymphatic anomaly, kaposiform lymphangiomatosis, and Gorham-Stout disease.
6. The method of any one of the preceding paragraphs, wherein the vascular anomaly is infantile hemangioma.
7. The method of any one of the preceding paragraphs, wherein the subject is further administered at least one beta-blocker, mTOR inhibitor, and/or steroid.
8. The method of paragraph 7, wherein the beta-blocker is selected from the group consisting of:
   R+ atenolol; atenolol; nadolol; propranolol; R+ propranolol; and timolol.
9. The method of paragraph 7, wherein the beta-blocker is R+ propranolol.
10. The method of paragraph 7, wherein the mTOR inhibitor is selected from the group consisting of:
   sirolimus; everolimus; temisrolimus; and rapamycin.
11. The method of paragraph 7, wherein the mTOR inhibitor is rapamycin.
12. The method of paragraph 7, wherein the steroid is a corticosteroid.
13. The method of paragraph 12, wherein the corticosteroid is selected from the group consisting of:
   dexamethasone; prednisone; prednisolone; triamcinolone; clobetasol propionate;
   betamethasone valerate; betamethasone dipropionate; and mometasone furoate.
14. A combination or kit comprising:
   at least one statin; and
   at least one beta-blocker, mTOR inhibitor, and/or steroid.
15. A combination or kit comprising:
   at least one statin; and
   at least one beta-blocker, mTOR inhibitor, and/or steroid;
   for use in treating a vascular anomaly.
16. A composition comprising at least one statin, for use in treating a vascular anomaly.
17. The combination, kit, or composition of one of the preceding paragraphs, wherein the statin is selected from the group consisting of:
   simvastatin; atorvastatin; cerivastatin; fluvastatin; lovastatin; mevastatin; pitavastatin;
   pravastatin; and rosuvastatin.
18. The combination, kit, or composition of one of the preceding paragraphs, wherein the statin is simvastatin.
19. The combination, kit, or composition of one of the preceding paragraphs, wherein the statin is atorvastatin.
20. The combination, kit, or composition of one of the preceding paragraphs, wherein the beta-blocker is selected from the group consisting of:
   atenolol; nadolol; propranolol; R+ propranolol; and timolol.
21. The combination, kit, or composition of one of the preceding paragraphs, wherein beta-blocker is R+ propranolol.
22. The combination, kit, or composition of one of the preceding paragraphs, wherein the mTOR inhibitor is selected from the group consisting of:
   sirolimus; everolimus; temisrolimus; and rapamycin.
23. The combination, kit, or composition of one of the preceding paragraphs, wherein the mTOR inhibitor is rapamycin.
24. The combination, kit, or composition of one of the preceding paragraphs, wherein the steroid is a corticosteroid.
25. The combination, kit, or composition of one of the preceding paragraphs, wherein the corticosteroid is selected from the group consisting of:
   dexamethasone; prednisone; prednisolone; triamcinolone; clobetasol propionate;
   betamethasone valerate; betamethasone dipropionate; and mometasone furoate.
26. The combination, kit, or composition of one of the preceding paragraphs, wherein the combination, kit, or composition of one of the preceding paragraphs is formulated for oral administration.
27. The combination, kit, or composition of one of the preceding paragraphs, wherein the combination, kit, or composition of one of the preceding paragraphs is a syrup or suspension.
28. The combination, kit, or composition of one of the preceding paragraphs, wherein the vascular anomaly is selected from the group consisting of:
   infantile hemangioma, congenital hemangioma, pyogenic granuloma, tufted angioma, kaposiform hemangioendothelioma, epitheloid hemangioendothelioma (EHE), capillary malformations, arteriovenous malformations, venous malformations, lymphatic malformations, complex lymphatic malformations, generalized lymphatic anomaly, central conducting lymphatic anomaly, kaposiform lymphangiomatosis, and Gorham-Stout disease.
29. The combination, kit, or composition of one of the preceding paragraphs, wherein the vascular anomaly is infantile hemangioma.

The technology described herein is further illustrated by the following examples which in no way should be construed as being further limiting.

### EXAMPLES

### Example 1

Hemangioma-derived stem cells (HemSC, n=6) were stimulated to undergo endothelial differentiation for 6 days and then treated with or without R+ propranolol for 2 hours. Total RNA was isolated from each sample for RNA Sequencing. Analysis revealed R+ propranolol in this setting inhibits eight enzymes in the mevalonate pathway, including 3 hydroxy-3-methylglutaryl-CoA reductase (HGMCR), the target of statins. From this, it was hypothesized that R+ propranolol inhibition of hemangioma vessel formation could be mimicked by inhibiting HGMCR using a statin such as simvastatin or atorvastin.

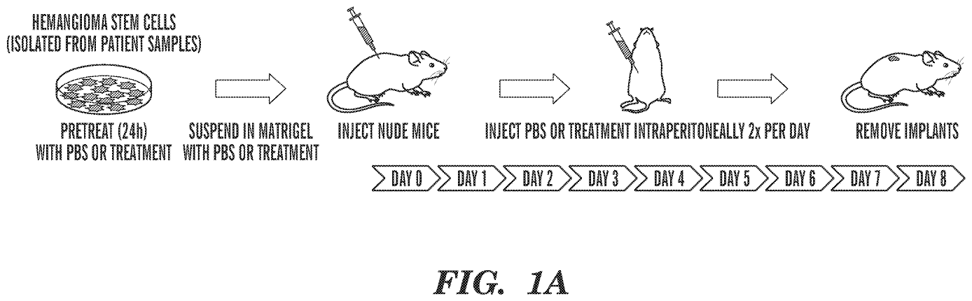

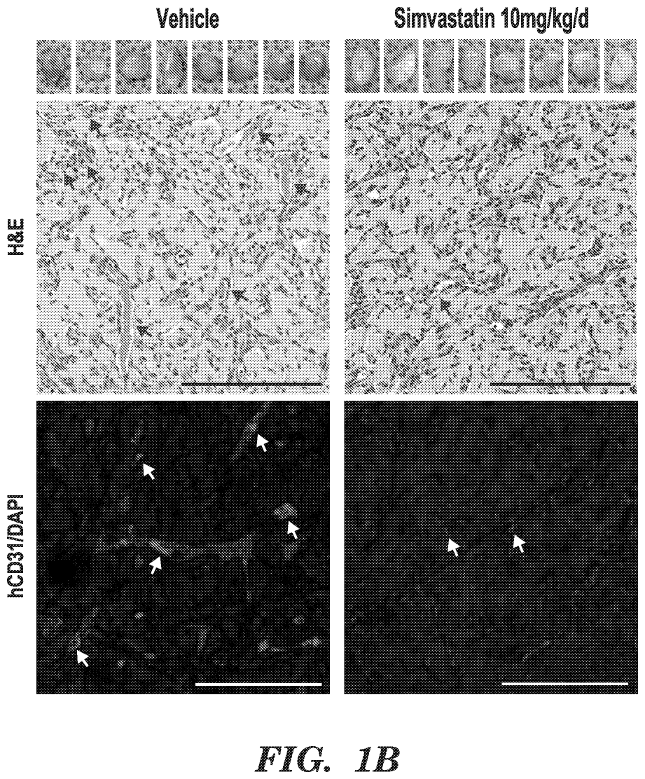

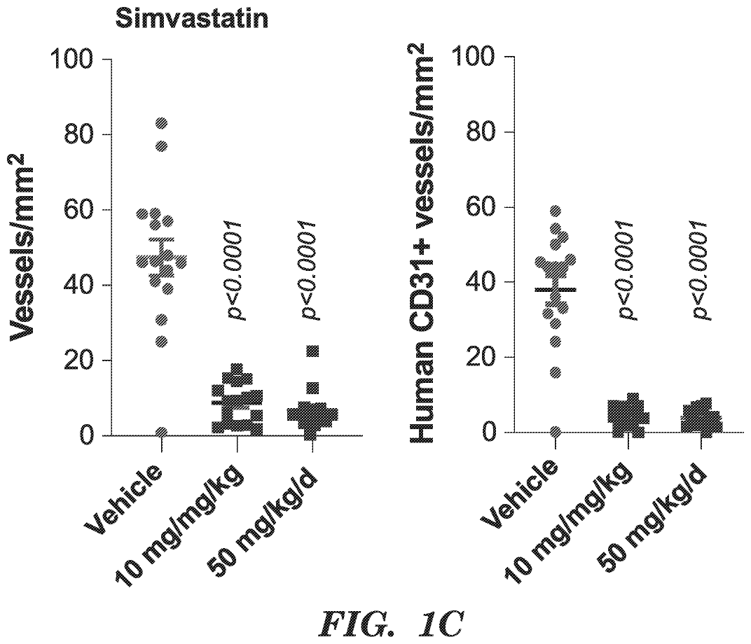

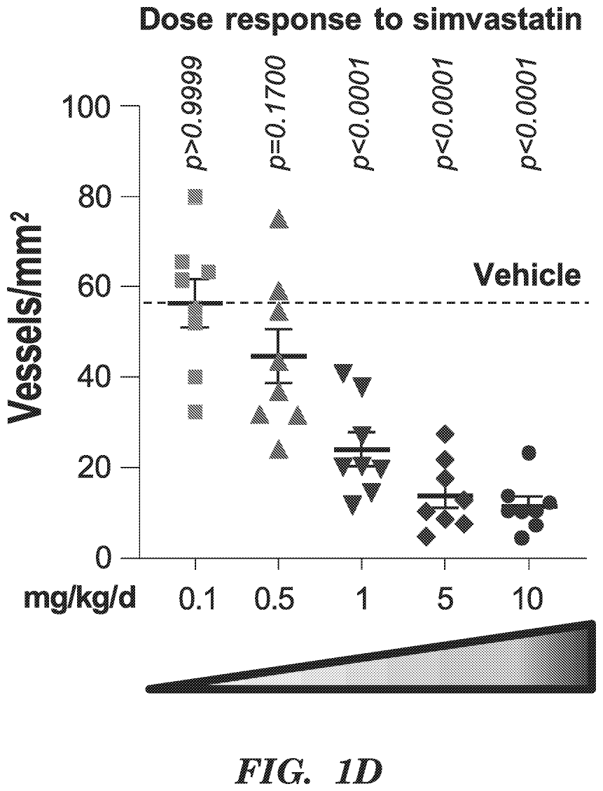

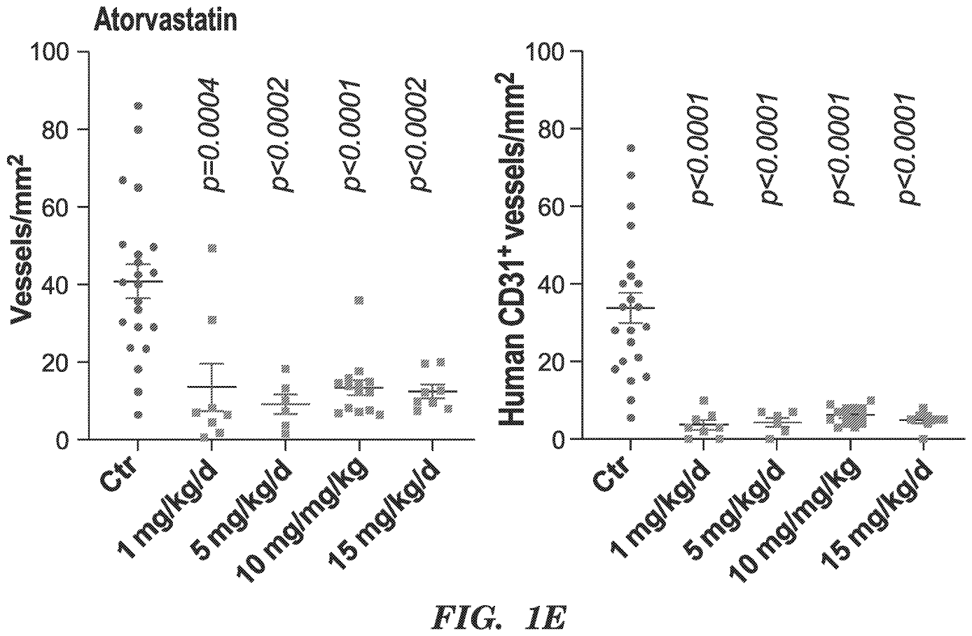

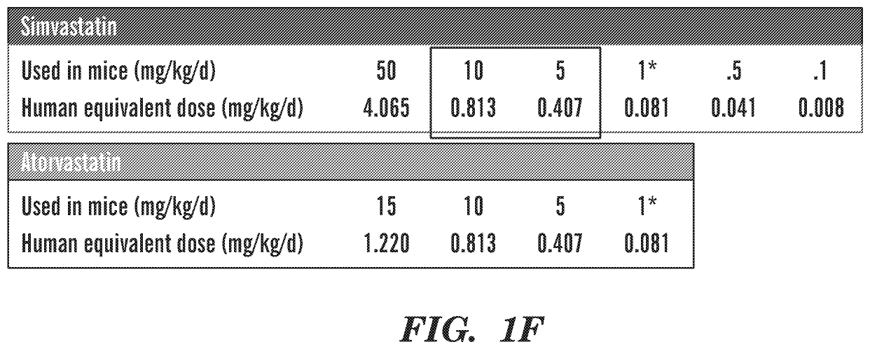

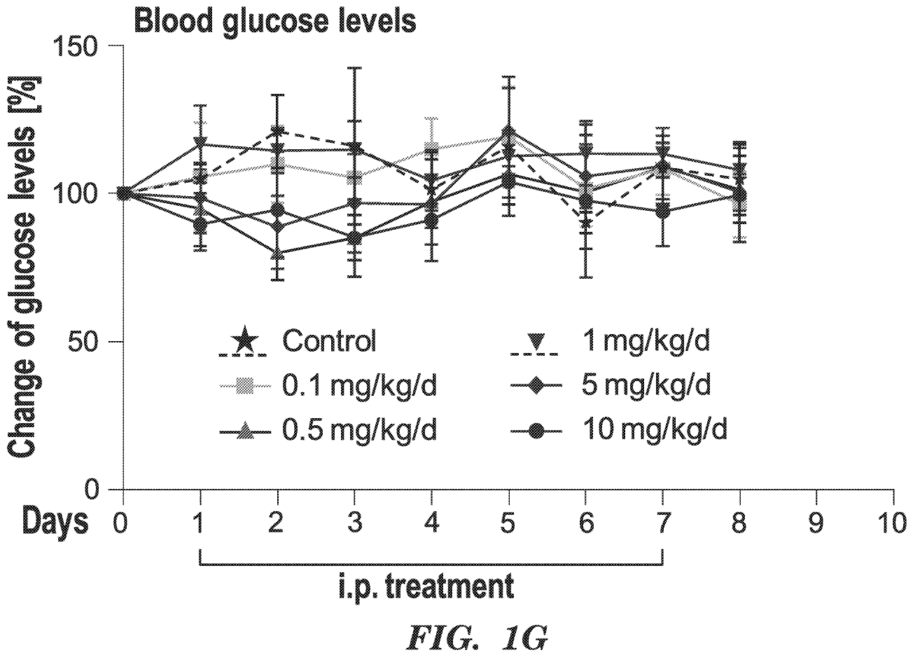

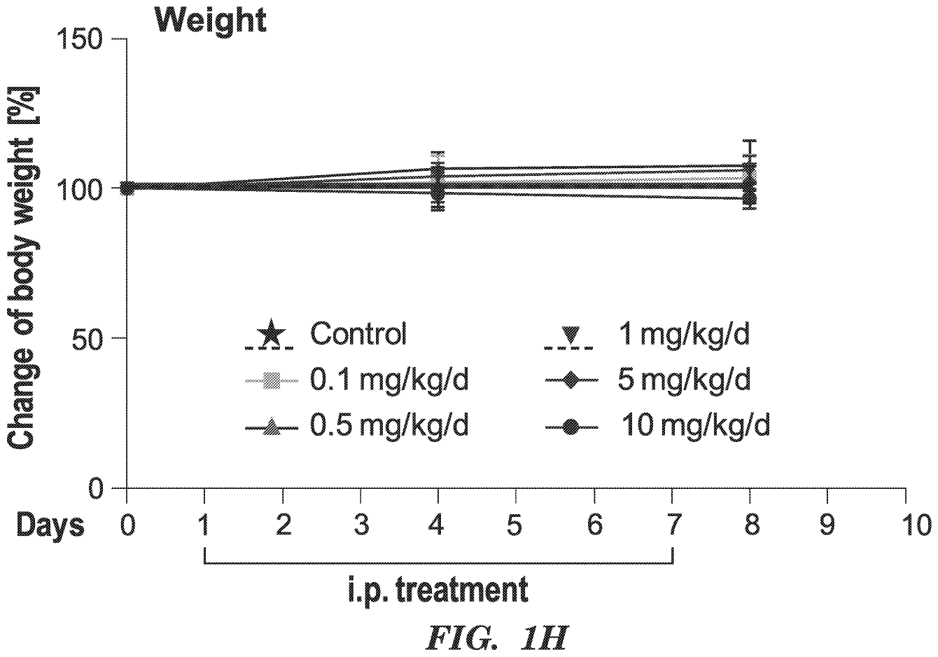

An in vivo xenograft model of infantile hemangioma, wherein HemSC are subcutaneously injected into into immune-deficient mice in a Matrigel suspension. The cells form. Tumors grew over the course of 7 days while the mice were treated with simvastan or atorvastatin via intraperitoneal injections. 1 mg/kg/d of simvastatin as well as atorvastatin was effective to significantly inhibit vessel formation (FIGS. 1A-1B) was used to test the effect of simvastatin (10 mg/kg/day) or atorvastatin (1 mg/kg/day) (FIG. 1A). Both statins significantly blocked the formation of human blood vessels from HemSC (n=3) (FIG. 1B). These results indicate that statins can block the vascular overgrowth that characterizes infantile hemangioma.

Targeting the mevalonate pathway with statins is a novel mechanism to block vascular overgrowth in infantile hemangioma, alone or in combination with known treatments. Hence, statins are contemplated herein as additive and/or alternative therapeutic option, e.g., in heavily affected infants with infantile hemangioma and for those infants who do not respond to propranolol.

### Example 2: Statins Repurposed: a SOX18-Mevalonate Pathway-Axis Drives Vascular Growth in Infantile Hemangioma

Propranolol is the mainstay treatment for infantile hemangioma (IH), the most common tumor in children. The R(+) enantiomer of propranolol, lacking β-adrenergic receptor antagonism, inhibits IH de novo vessel formation via suppression of SRY box 18 (SOX18) transcription factor activity. Here, transcriptomic profiling of patient-derived hemangioma stem cells (HemSC) undergoing endothelial differentiation and treated with or without R(+) propranolol revealed a coordinated downregulation of genes in the mevalonate pathway (MVP). Loss of function experiments confirmed SOX18-mediated inhibition of the MVP by R(+) propranolol. MVP inhibitors, simvastatin and atorvastatin, inhibited blood vessel formation in a preclinical xenograft model with patient-derived HemSC. Moreover, evidence for MVP activation was detected in proliferating phase IH tissue. Described herein is a novel theranostic approach to repurpose statins for the treatment of IH based on a SOX18-MVP-axis as a central regulator in IH pathogenesis.

### Introduction:

Infantile hemangioma (IH) is the most common pediatric tumor and serves as a paradigm for vasculogenesis, angiogenesis, and vascular regression¹. It is a benign vascular tumor with an incidence of 2-10% and predominantly occurs in female and premature infants of European descent. IH follows a unique life cycle: it arises postnatally at 3-7 weeks with rapid growth during the proliferating phase, which can continue for 12 months. A spontaneous and gradual involuting phase follows that spans 2-7 years. While for most children, IH poses no serious risk, 10-15% of lesions are considered complex and require treatment. Propranolol was discovered to be effective for IH². Propranolol is currently the only FDA-approved drug for IH, based on results from a randomized controlled clinical trial³. Despite its successful repurposing for IH, propranolol is associated with side effects in infants including hypotension, bradycardia, peripheral vasospasm, hypoglycemia and seizures, bronchospasm, slowed weight gain, diarrhea, agitation, sleep disturbance, and negative neurocognitive long-term outcomes^{3 4-6}. Further, the complete response rate to propranolol was reported to be 60%³. The safety and efficacy concerns, underscore the need for alternative treatment for infants with IH.

It is firmly established that propranolol acts as non-selective antagonist of the GPCR β1- and β2-adrenergic receptors but of note is that propranolol consists of an equimolar (1:1) mixture of S(−) and R(+) enantiomers. The S(−) enantiomer is a potent antagonist of β1 and β2-adrenergic receptors, while the R(+) enantiomer is largely devoid of beta-blocker activity⁷. This provided an opportunity to test the non-beta blocker R(+) enantiomer for β1 and β2-adrenergic receptor-independent effects on hemangioma stem cells (HemSC), which have been isolated from proliferating phase IH specimens and shown to recapitulate hemangiogenesis in nude mice⁸. R(+) propranolol inhibits HemSC endothelial differentiation in vitro and HemSC vasculogenesis in vivo. Both R(+) propranolol and R(+) atenolol inhibit the transcription factor SRY (sex-determining region Y) HMG box-containing 18 (SOX18) activity, therefore preventing HemSC differentiation and disease onset and progression in a pre-clinical model in vivo. Importantly this pharmacological interference has been observed with Sm4—a small compound blocker of the transcription factor (TF) SOX18, further validating the role of this molecular target in IH^{9,10}. Described herein is the identification of the molecular basis of the mechanism of action of propranolol in IH that is mediated by a SOX18-dependent inhibition of HemSC endothelial differentiation and vessel formation in vivo^{9,10}.

The SOX18 gene is a master regulator of vascular development and differentiation and is expressed in nascent blood and lymphatic endothelium as well as in endothelial progenitor cells in adults¹¹. It plays fundamental roles in arterial specification¹², lymphangiogenesis¹³ and tumor angiogenesis¹⁴. Its pharmacological blockade has prompted us to investigate the molecular basis involved in HemSC endothelial differentiation.

To further elucidate the functional role of SOX18 in IH vasculogenesis, genes whose expression in differentiating HemSC are altered by R(+) propranolol were identified. It was discovered that R(+) propranolol coordinately downregulates transcripts encoding enzymes in the mevalonate pathway (MVP). The MVP is central to cholesterol and isoprenoid biosynthesis and is controlled by the rate-limiting enzyme 3-hydroxy-3-methylglutaryl-coenzyme reductase A (HMGCR)¹⁹, which produces mevalonate. Additional regulators are the transcription factor sterol regulatory element binding protein-2 (SREBP2) that induces expression of MVP genes²⁰ and ATP-binding cassette transporter A1 (ABCA1) that acts as a negative regulator of SREBP2 by facilitating retrograde transport of cholesterol from the plasma membrane to the endoplasmic reticulum (ER)²¹. Statins, competitive HMGCR inhibitors, are widely prescribed to reduce low-density-lipoprotein cholesterol in patients at risk for cardiovascular disease²². Pleiotropic activities of statins beyond the lipid-lowering effect have been established²³⁻²⁵ Accumulating evidence suggests a role for statins in epigenetic modifications in a cardiovascular disease and an oncology context²⁶⁻²⁸. Described herein is a novel molecular relationship between an endothelial specific TF and the MVP. It is demonstrated that that R(+) propranolol causes a SOX18-mediated, global downregulation of MVP genes in HemSC during their endothelial differentiation process, indicating that statins can be safely repurposed to treat the vascular overgrowth in IH.

### Results:

### R(+) Propranolol Globally Reduces Transcript Levels of MVP Genes in Patient-Derived HemSC Undergoing Endothelial Differentiation

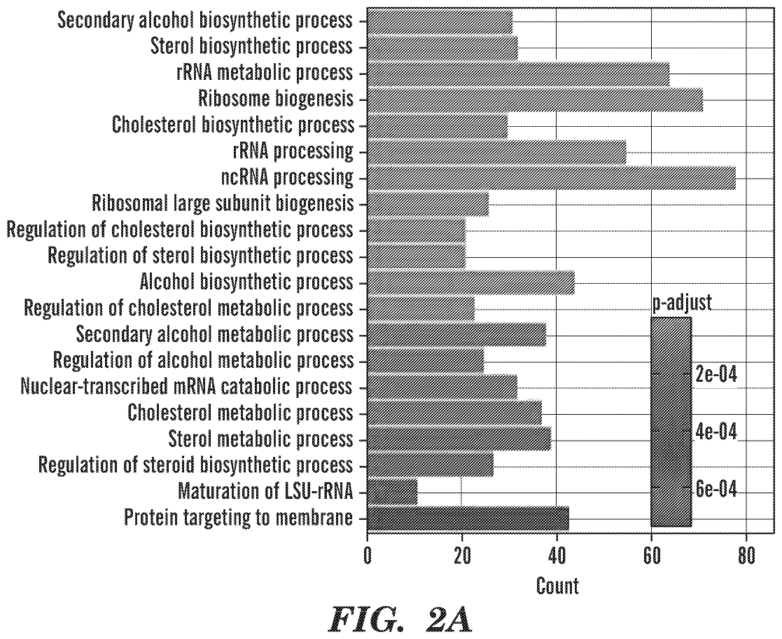

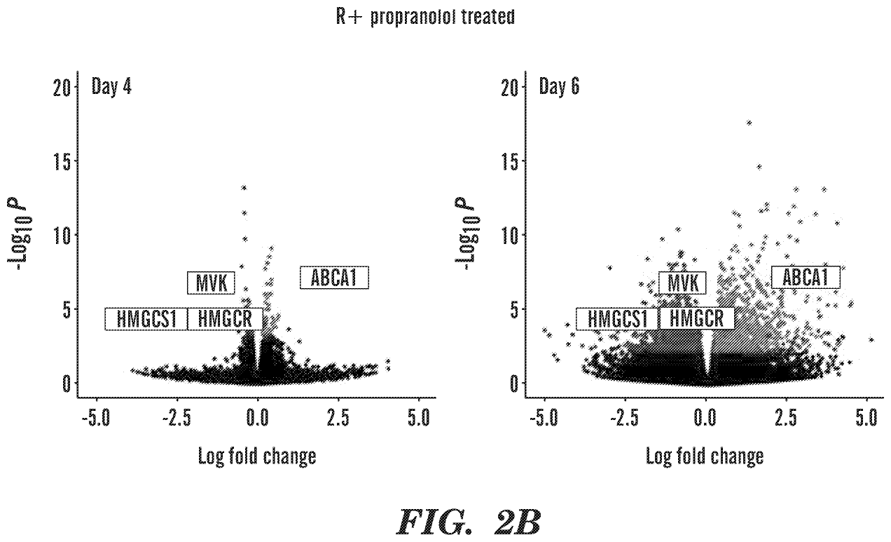

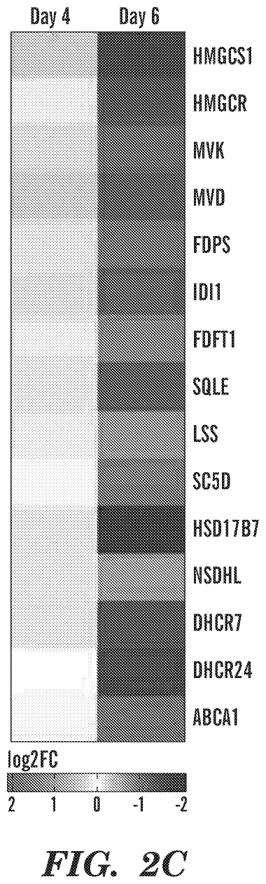

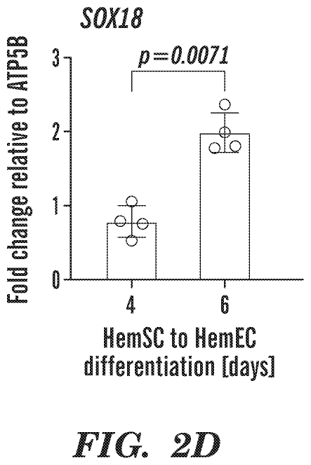

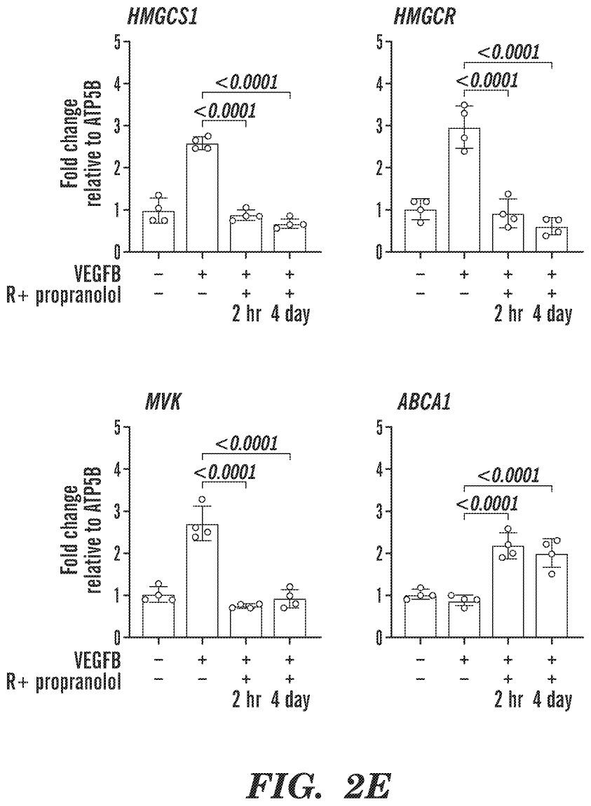

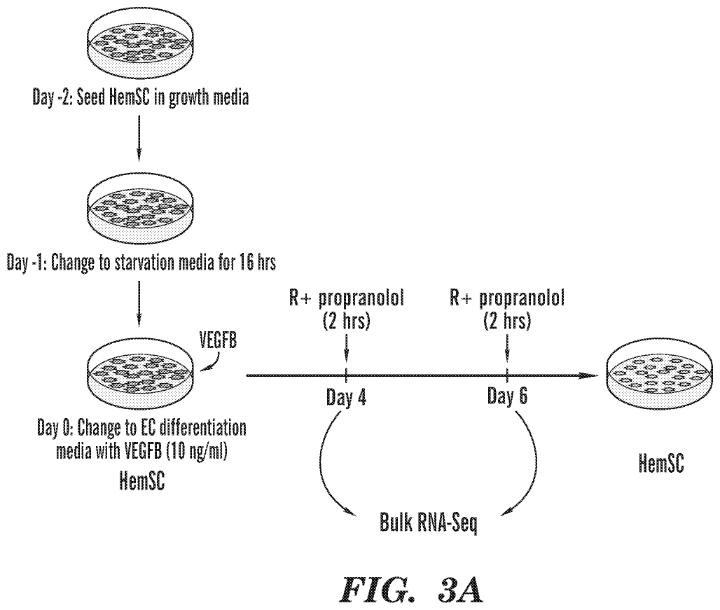

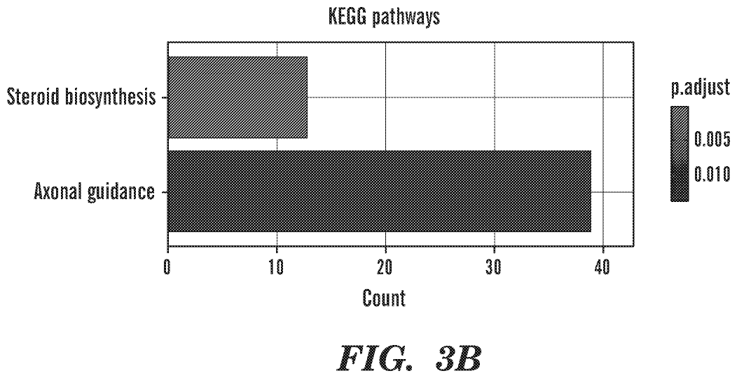

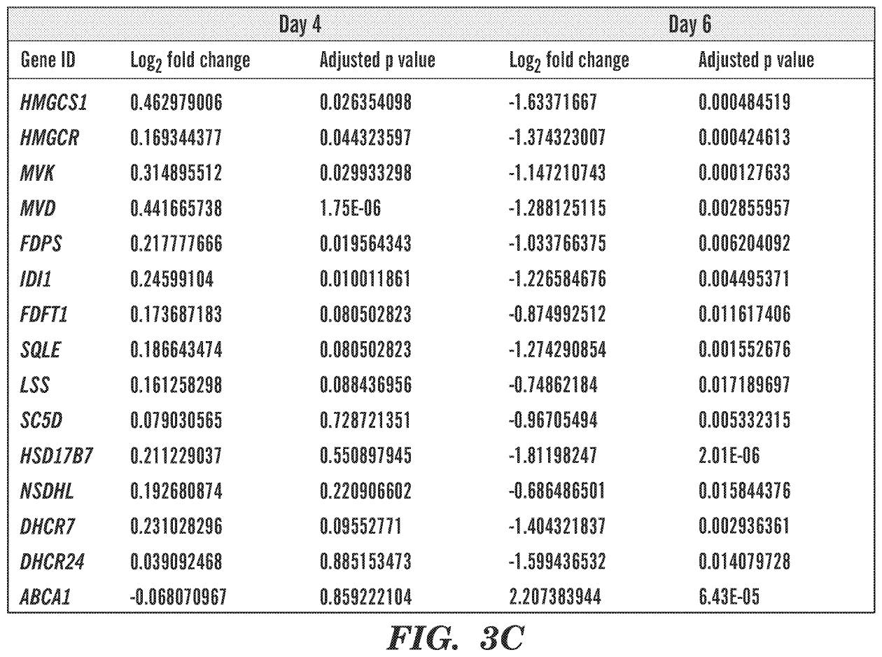

To identify the downstream targets of R(+) propranolol in HemSC to hemangioma endothelial cell (HemEC) differentiation in a non-biased way, bulk RNA sequencing of HemSC isolated from 6 different IH specimens was performed. The HemSCs were induced to undergo endothelial differentiation for 6 days and then treated with or without R(+) propranolol (20 μM) for 2 hours (FIG. 3A). The timing and dosage were determined by previously observed downregulation of NOTCH1 expression, a widely known SOX18 transcriptional target, under this treatment condition¹⁰. HemSC at Day 4 of endothelial differentiation, prior to onset of endothelial marker expression, were analyzed as well. The Kyoto Encyclopedia of Genes and Genomes (KEGG) analysis identified steroid biosynthesis as differentially affected (FIG. 3B), which was confirmed by gene ontology analysis of the subontology biological processes (FIG. 2A). Subsequent gene by gene analysis revealed R(+) propranolol treatment on Day 6 significantly reduced transcripts encoding several enzymes of the MVP, including the rate-limiting enzyme HMGCR as well as HMGCS1 and MVK (FIGS. 2B, 2C). The significant increase in SOX18 mRNA from Day 4 to Day 6 of differentiation is consistent with the onset of R(+) propranolol sensitivity at Day 6 (FIG. 2D). Downregulation of HMGCS1, HMGCR, and MVK by treatment with R(+) propranolol for 2 hours or 4 days was confirmed in independent experiments. ABCA1, a negative regulator of SREBP2 activity, was upregulated by 2 hours or 4 days treatment with R(+) propranolol (FIG. 2E). There were 105 differentially regulated genes in HemSC at Day 4 of differentiation versus 2482 at Day 6 of differentiation. Differential expression was defined as log 2 fold change >1 and adjusted p-value <0.05. This defines a critical time window for SOX18 inhibition and therefore R(+) treatment in the HemSC differentiation process. This notion is further supported by the downregulation of MVP transcripts that was not seen in HemSC at Day 4 of differentiation, prior to endothelial marker expression.

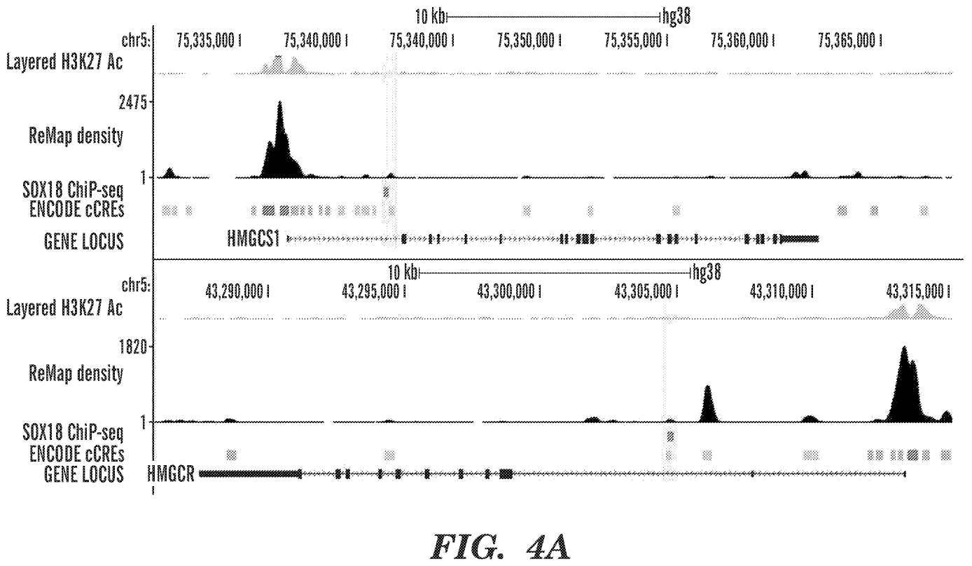

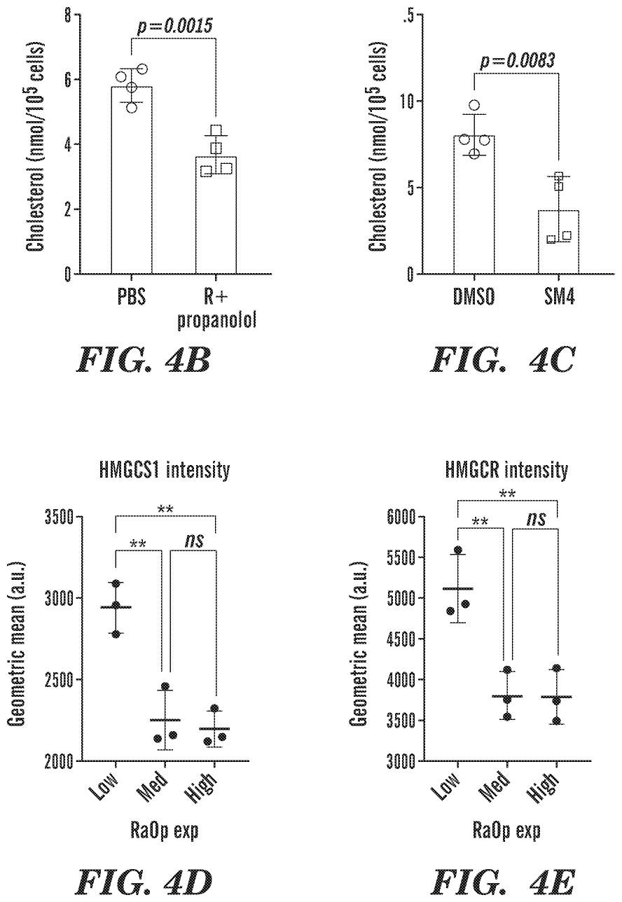

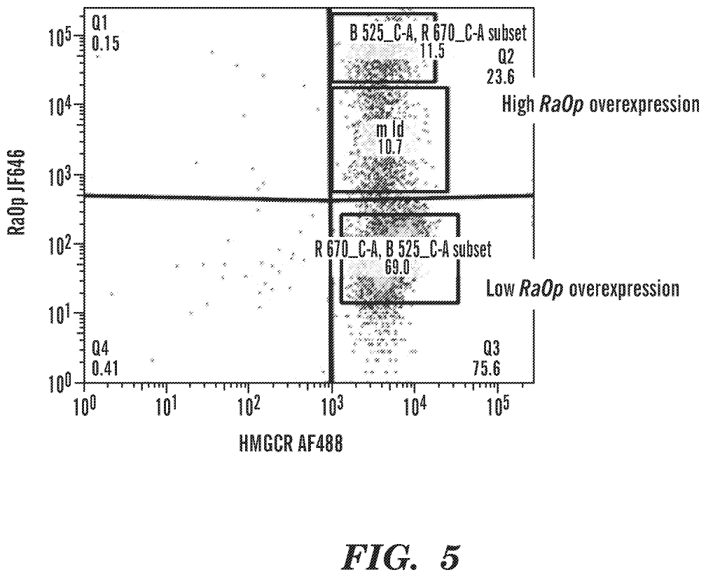

To further examine the effect of SOX18 inhibition on the MVP, SOX18 binding locations in the genome were investigated. Using a publicly available ChIP-seq dataset in HUVEC (Overman et al elife 2017) SOX18 binding locations were found within the HMGCR and HMGCS1 gene loci. The SOX18 binding locations also correspond with ENCODE candidate cis-regulatory elements (FIG. 4A). These experiments, combined with the identification of SOX18 binding sites in regulatory regions of HMGCS1 and HMGCR, establish the ability of R(+) propranolol to downregulate critical genes in or related to the MVP, most likely through a SOX18-dependent mechanism.

### SOX18 Activity Positively Regulates the MVP in Normal Endothelial Cells

To validate the role of SOX18 in regulating genes in the MVP, we tested whether pharmacological disruption of SOX18 activity through treatment with R(+) propranolol or the known SOX18 inhibitor Sm⁴²⁹ would have an effect on cholesterol biosynthesis. Human umbilical vein endothelial cells (HUVEC) were cholesterol depleted by incubation with methyl beta-cyclodextrin (MBCD) and then treated ±R(+) propranolol or ±Sm4, for 16 hours, allowing for new cholesterol synthesis, measured by mass spectrometry. Endogenous cholesterol levels were significantly reduced in both R(+) propranolol-treated and Sm4-treated HUVECs, consistent with SOX18-mediated downregulation of MVP gene expression (FIGS. 4B-4C). Conversely, we tested the effect of an overexpressed, dominant negative version of SOX18 (Ragged Opossum (RaOp)) that is known to disrupt SOX18 activity³⁰, using the fluorescently tagged SOX18^{RaOp} expressed in HUVECS. High expression of SOX18^{RaOp} decreased immunofluorescent staining for HMGCS1 and HMGCR, compared to low SOX18^{RaOp} indicating a gene dose response effect on the two key MVP enzymes (FIGS. 4D-4E). Taken together both experiments demonstrate that SOX18 activity positively regulates cholesterol biosynthesis during IH differentiation.

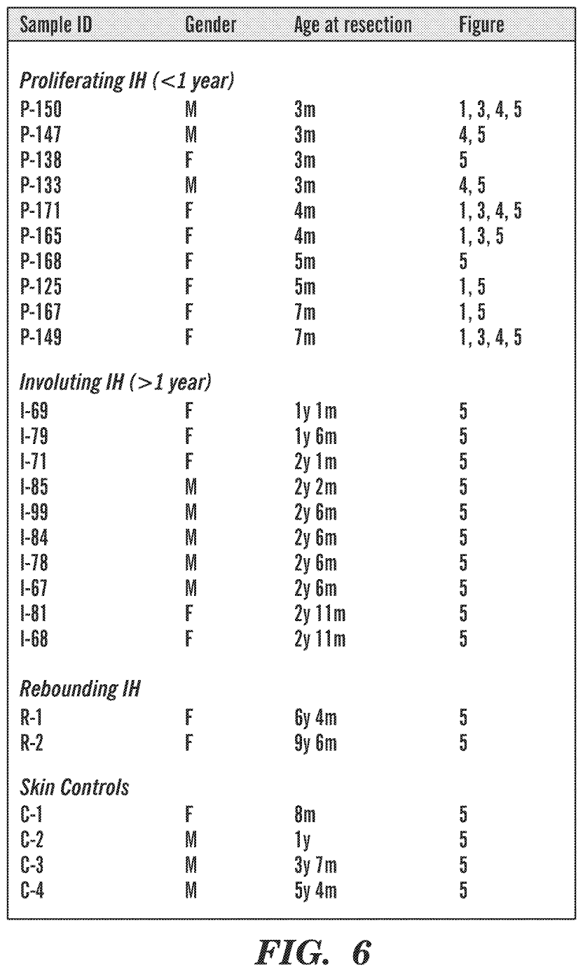

FIG. 6 provides an overview of IH tissues used throughout this study for HemSC and HemEC isolation and FFPE tissue sections for immunostaining; the far-right column denotes the figures the respective cells or tissues were included in.

### R(+) Propranolol Reduces the Mature Form of SREBP2, a Master Regulator of MVP Genes

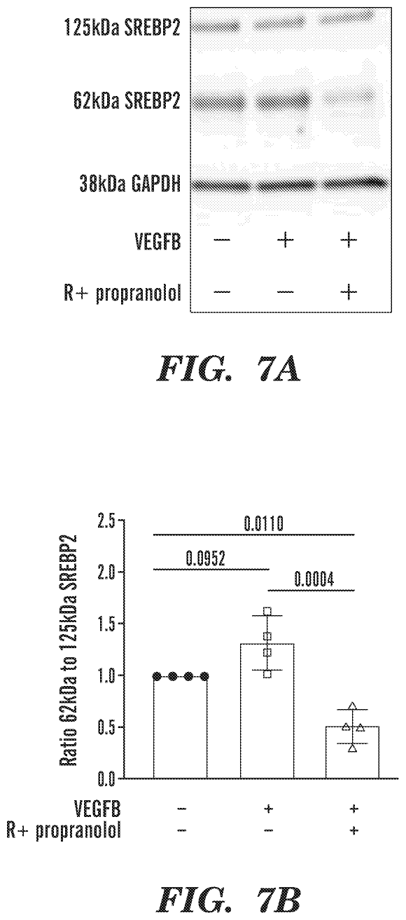

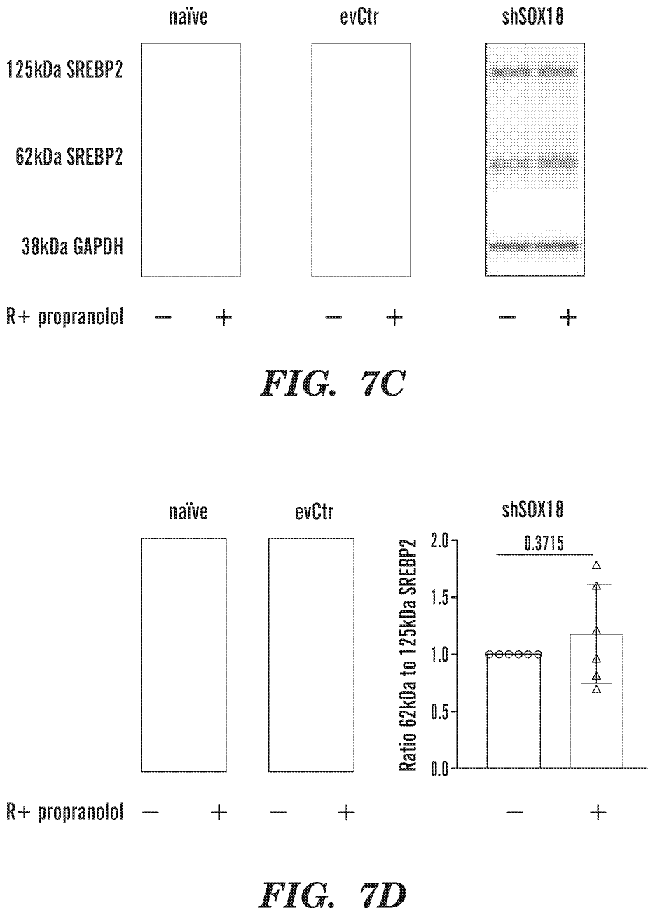

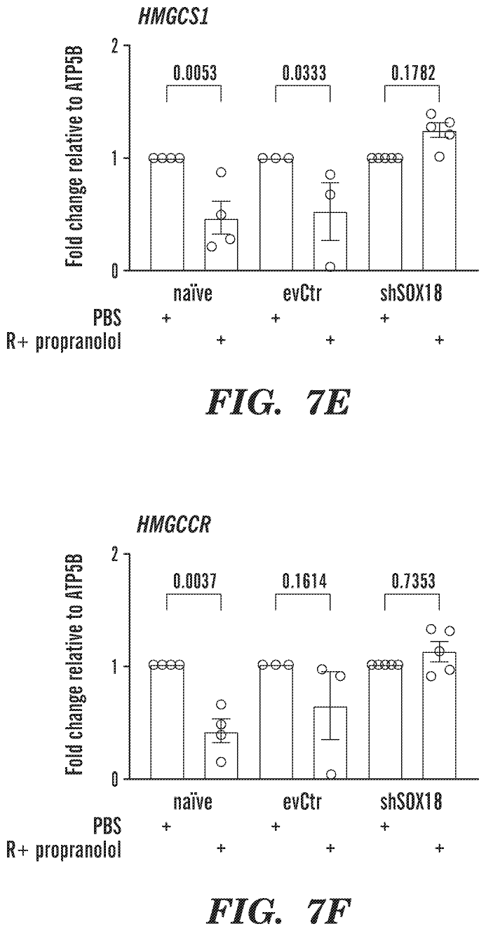

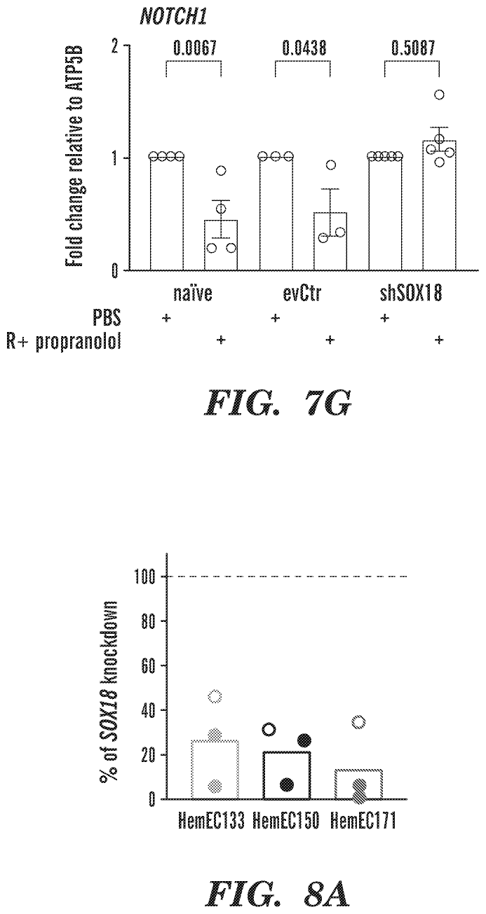

To further elucidate whether R(+) propranolol affects the MVP at the level of its well characterized transcriptional effector regulator, its effect on levels of the precursor (inactive) 125 kDa form of SREPB-2, which is anchored in the endoplasmic reticulum, and the 62 kDa mature form^{20,31} were analyzed. The 62 kDa mature form is proteolytically cleaved from the 125 kDa precursor when cholesterol levels drop, signaling the need for new biosynthesis. The 62 kDa mature form is the NH₂ basic helix loop helix (bHLH) leucine zipper domain that moves into the nucleus to activate transcription. Thus, it was hypothesized that R(+) propranolol treatment would reduce the 62 kDa mature form in favor of the 125 kDa precursor form of SREBP2 in HemSC undergoing endothelial differentiation. HemSC (n=4) were treated for 2 hours ±R(+) propranolol on Day 6 of VEGF-B induced endothelial differentiation. Cell lysates analyzed by Western Blot (WB) showed that R(+) propranolol decreased mature 62 kDa SREBP2 and decreased the 62 kDa/125 kDa ratio (FIGS. 7A, 7B). This is consistent with transcriptomic findings from bulk RNA-seq and qPCR. Rapid turnover of mature SREBP2 within 4 hours has been previously demonstrated³³.

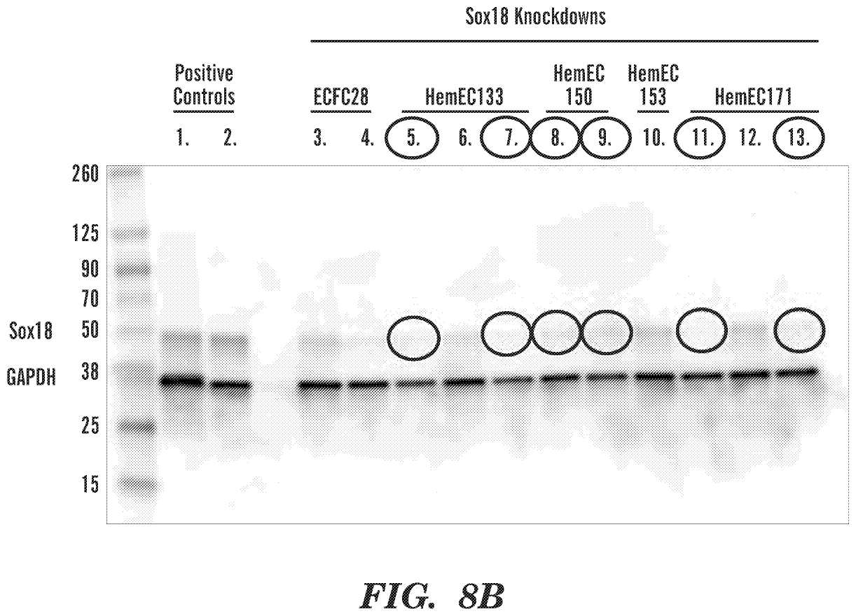

It was next assessed whether SOX18-dependency of the R(+) propranolol mediated effects by genetic knockdown of SOX18 using shRNA. Lentiviral knockdown of SOX18 in three different IH-derived HemEC was conducted and the extent of SOX18 knockdown was verified by qPCR and WB (FIGS. 8A, 8B). Knockdown of SOX18 in HemEC abolished sensitivity of SREBP2 to R(+) propranolol demonstrated by a constant ratio of 62 kDa to 125 kDa following ch22 R(+) propranolol treatment (FIGS. 7C, 7D). The requirement for SOX18 was further verified by qPCR measurement of mRNA transcript levels for SREBP2 target genes HMGCS1 and HMGCR. R(+) propranolol did not reduce the mRNA levels in shSOX18 HemEC (FIG. 7E, 7F). The known SOX18 target NOTCH1 served as a positive control for R(+) propranolol (FIG. 7G). The lentiviral SOX18 knockdown was performed independently three times in each of the three HemEC (denoted as HemEC 171, 150, and 133). Taken together, the decrease in expression of HMGCS1 and HMGCR and the reduction in mature 62 kDa SREBP2 by both pharmacological and genetic disruption of SOX18 demonstrates a functional link between SOX18, SREBP2, and the MVP.

### Statins Inhibit Vessel Formation in a Xenograft Model of IH

It was hypothesized that if the MVP is critical to IH onset and progression, statins may inhibit HemSC blood vessel formation in an IH preclinical model. The effect of statins on patient-derived cells has not been reported before and it was therefore first tested for cell toxicity and efficacy of statins on HemSC. Simvastatin and atorvastatin were used in this preclinical model as they are the two most commonly used statins in patients, including infants in the case of simvastatin³⁴. It is well established that atorvastatin is 5-10 times more potent than simvastatin (Jones P, Am J Cardiol, 1998). To first address toxicity, HemSC (n=3) were induced to undergo endothelial differentiation until day 6 while treated with simvastatin (0.1-1 μM) or atorvastatin (0.01-0.1 μM) and respective vehicle controls. Neither statin had an effect on differentiating HemSC viability compared to vehicle controls (FIG. 8A). Next, the effect of statins on LDL-receptor levels in HemSC were tested as an indirect measure of their effect on the MVP. Treatment with 5 μM simvastatin or 1 μM atorvastatin for 24 hours resulted in a significant upregulation of LDL-R mRNA in HemSC (n=4) (FIG. 8B), indirectly indicating efficient inhibition of HMGCR.

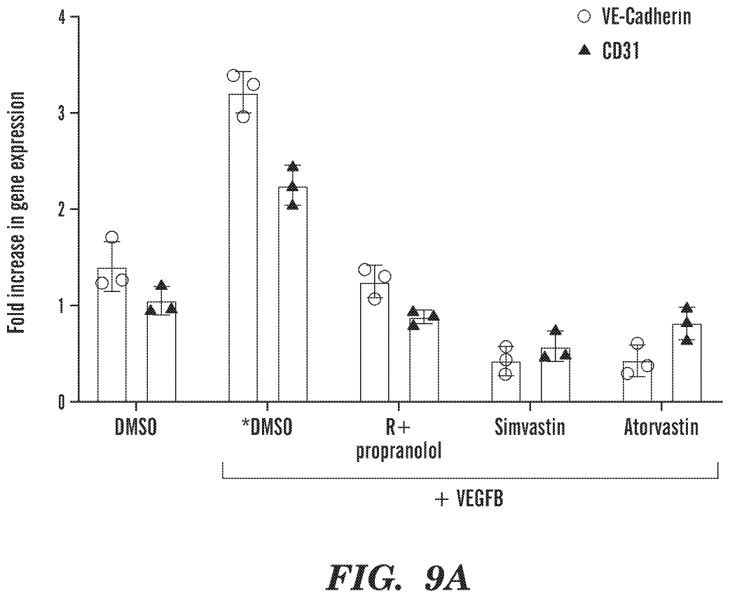

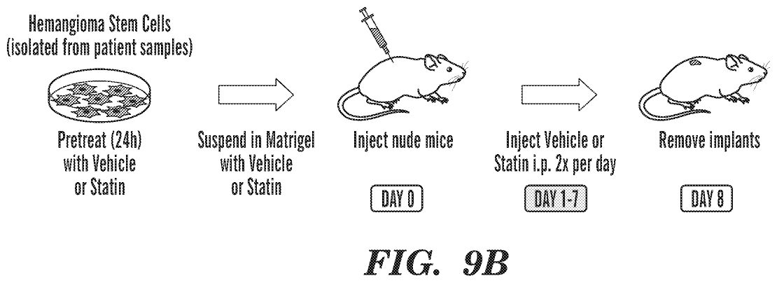

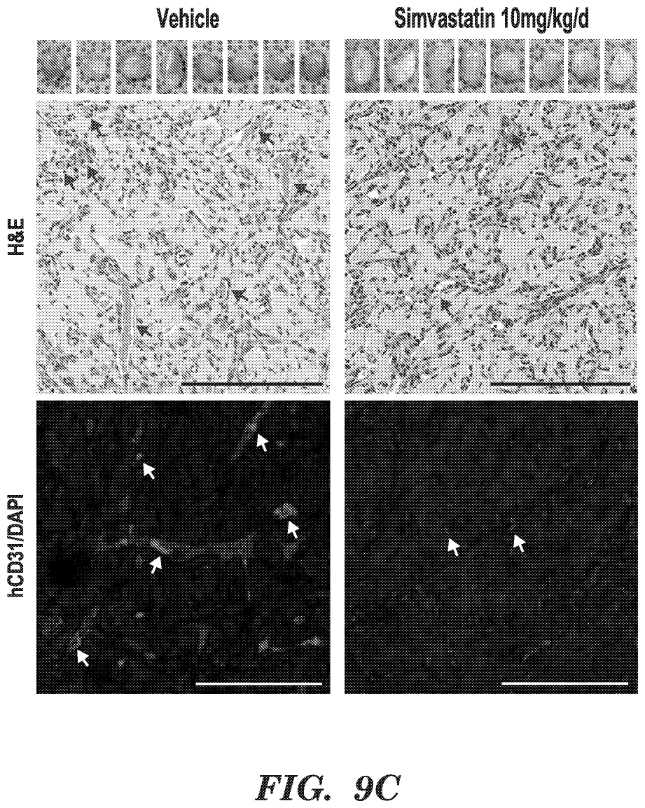

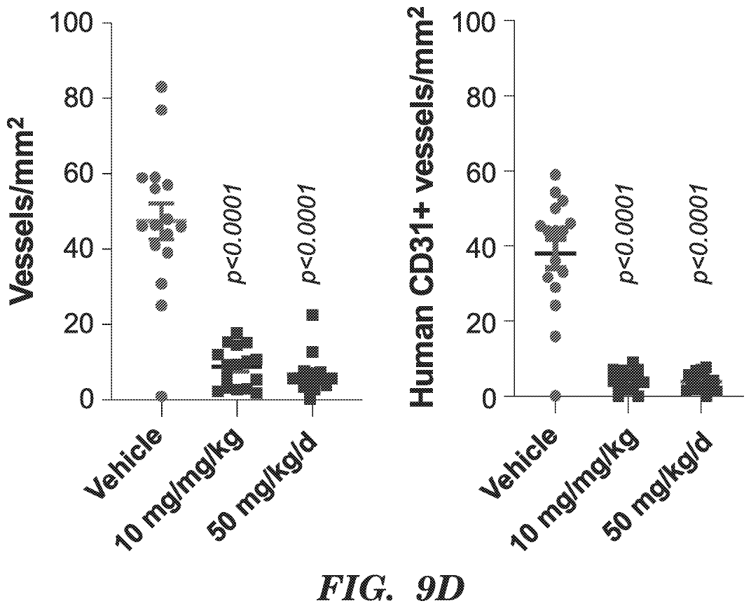

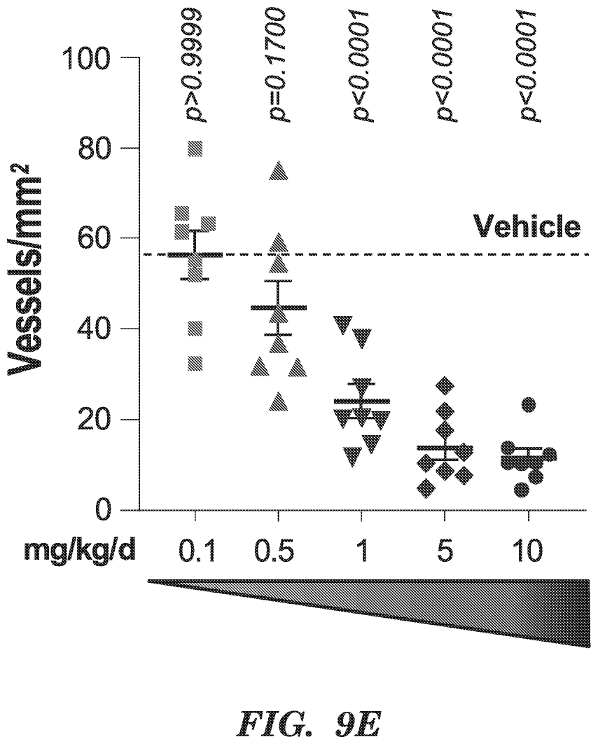

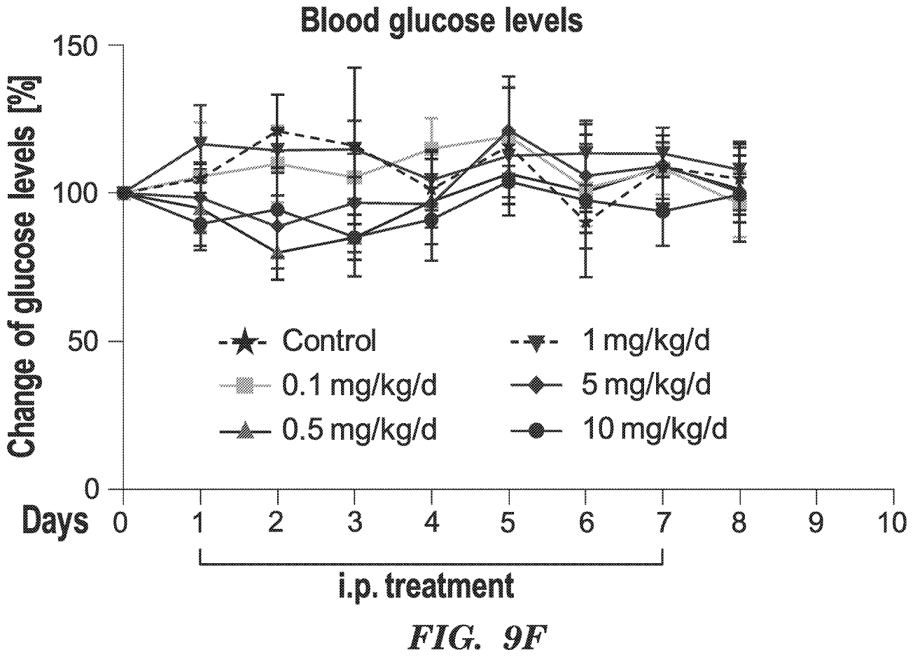

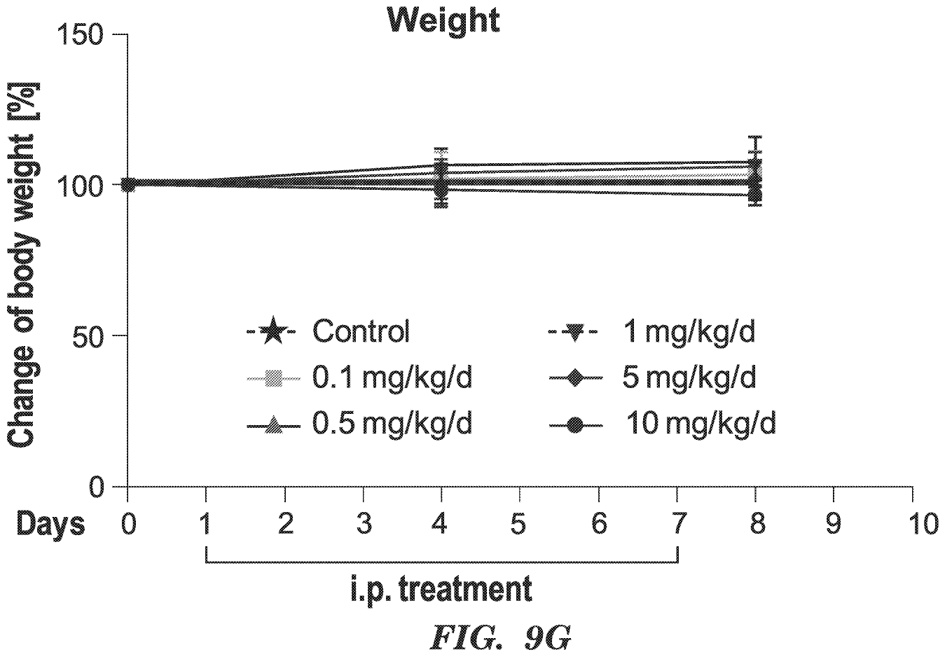

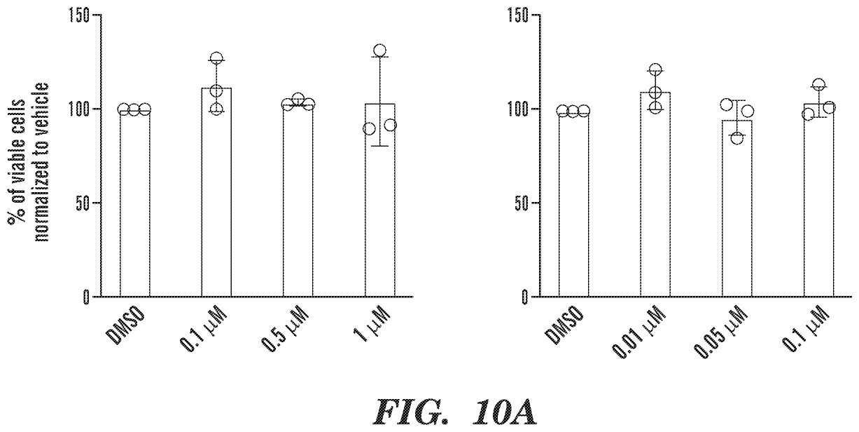

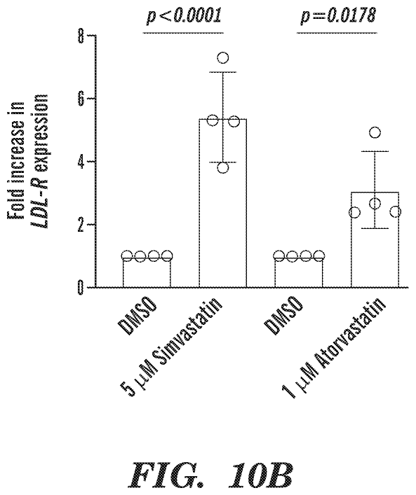

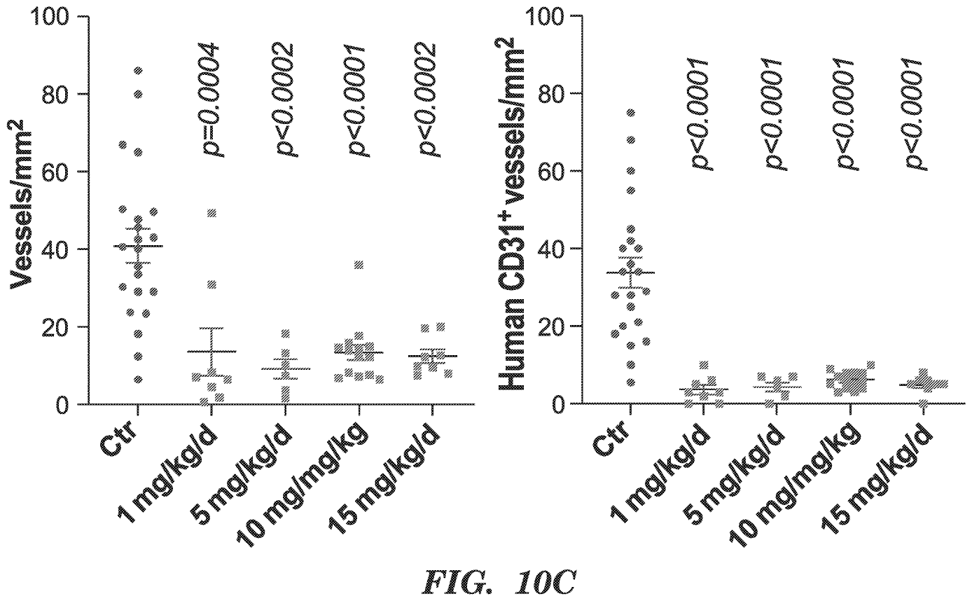

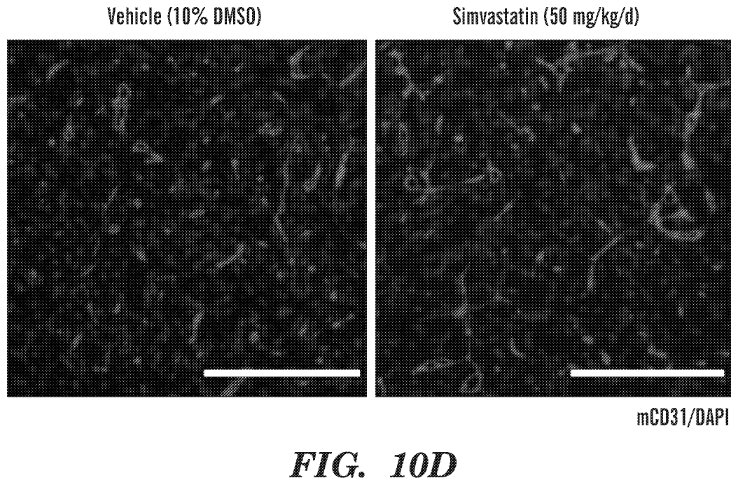

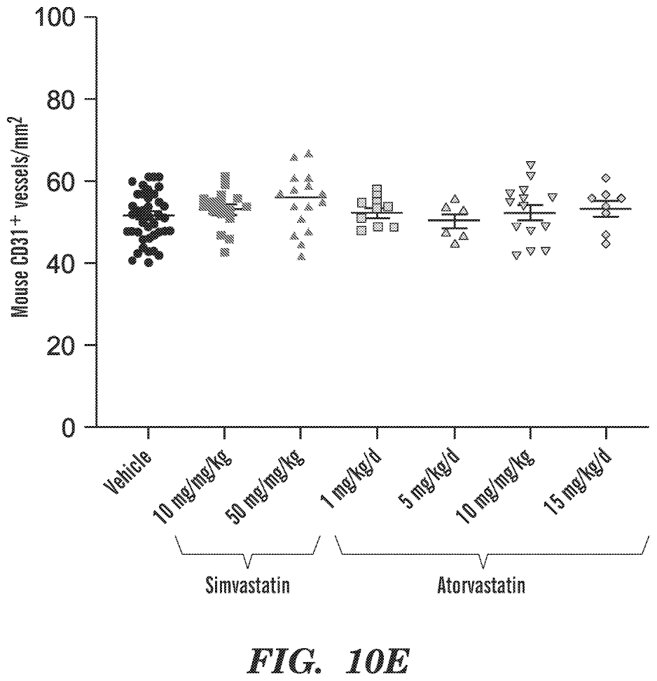

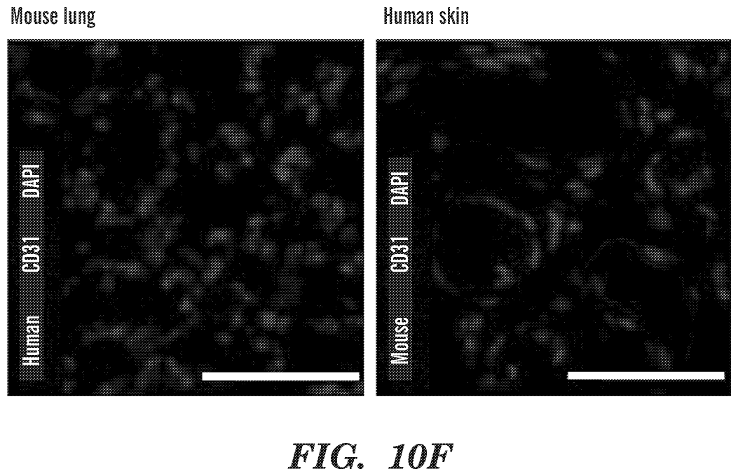

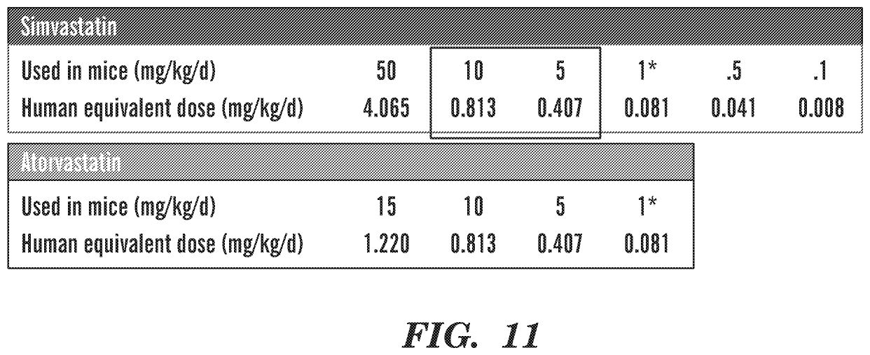

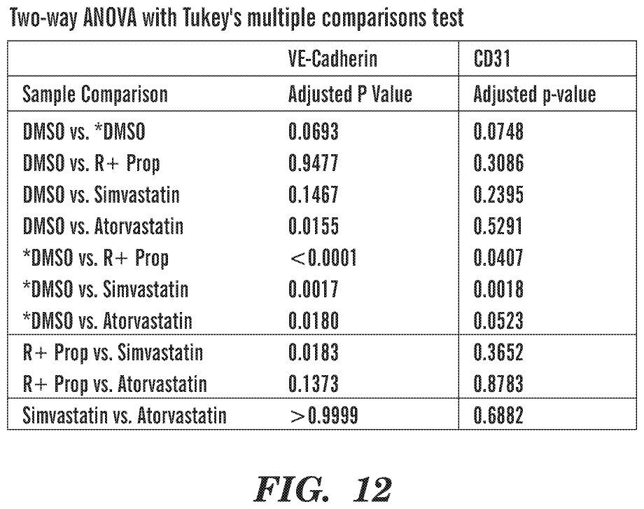

To determine if statins affect IH vasculogenesis, 1 μM simvastatin and 0.1 μM atorvastatin were tested on VEGF-B-induced HemSC endothelial differentiation as described⁹. Both simvastatin and atorvastatin significantly inhibited endothelial differentiation as indicated by decreased expression of the EC markers CD31 and VE-Cadherin compared to vehicle control (n=3). R(+) propranolol was included as a positive control (FIG. 9A). It was next tested if statins would impact de novo vessel formation in the murine xenograft model using IH-derived HemSC (n=4 different patients) (FIG. 9B). Simvastatin at 10 mg/kg/d (FIGS. 9C, 9D) and atorvastatin at 1 mg/kg/d (FIG. 10C) both significantly inhibited human CD31+ blood vessel formation. A simvastatin dose response experiment further showed that 1 mg/kg/d was sufficient to significantly inhibit vessel formation (FIG. 9E). Glucose levels and body weight of mice in the simvastatin dose response experiment were unaffected (FIGS. 9F, 9G). The human equivalent doses corresponding to the doses used in mice are shown in FIG. 11. The effective doses of simvastatin and atorvastatin in FIG. 7 are below effective doses used in adults and significantly below the dose used in infants for Smith Lemli Opitz Syndrome (0.5-1 mg/kg/d simvastatin) 34. Of note, the inhibitory effect of statins was limited to HemSC de novo vessel formation and did not impact angiogenic sprouting and ingrowth of surrounding murine vessels into the Matrigel implant (FIG. 10D). Antibody specificity for anti-human and anti-mouse CD31 was tested (FIG. 10E). Taken together, this data demonstrates that simvastatin and atorvastatin inhibit endothelial differentiation of HemSC in vitro and vasculogenesis in a preclinical IH xenograft model. This indicates the MVP contributes to vascular overgrowth in IH and can be effectively targeted by statins in a translational approach.

### Nuclear SOX18 and SREBP2 in Proliferating Phase and Rebounding IH Indicate Active MVP.

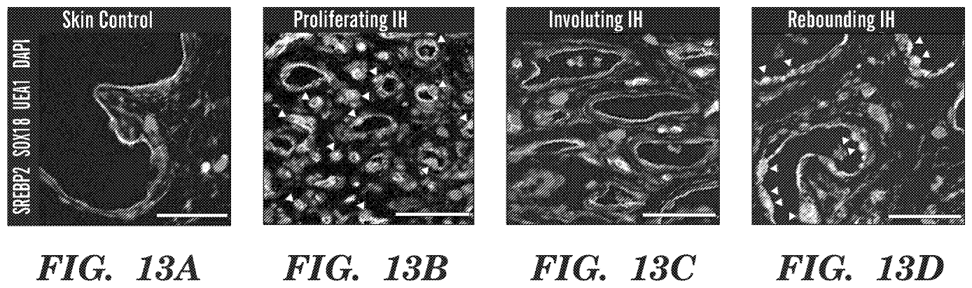

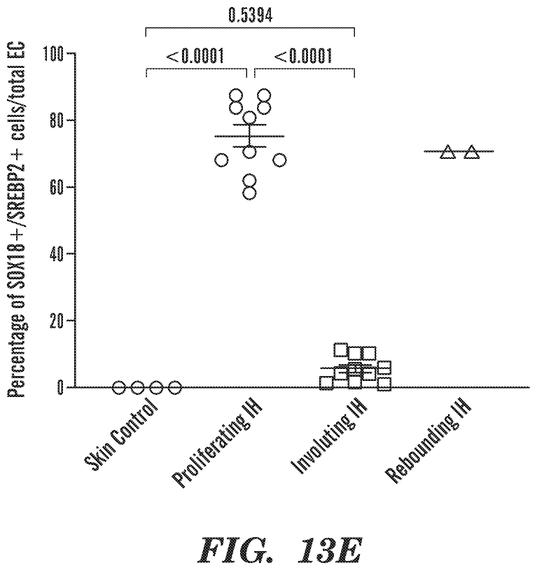

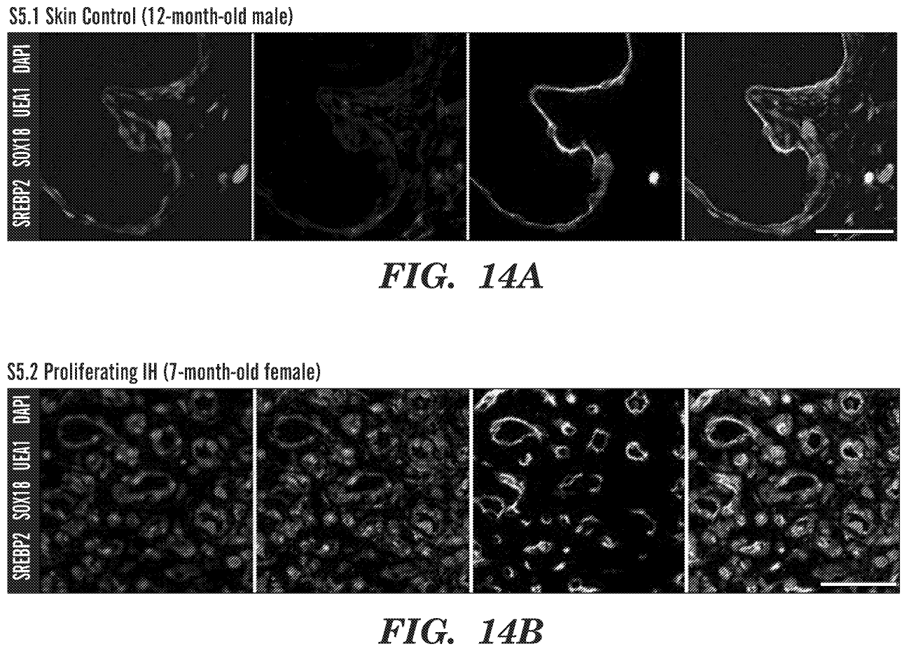

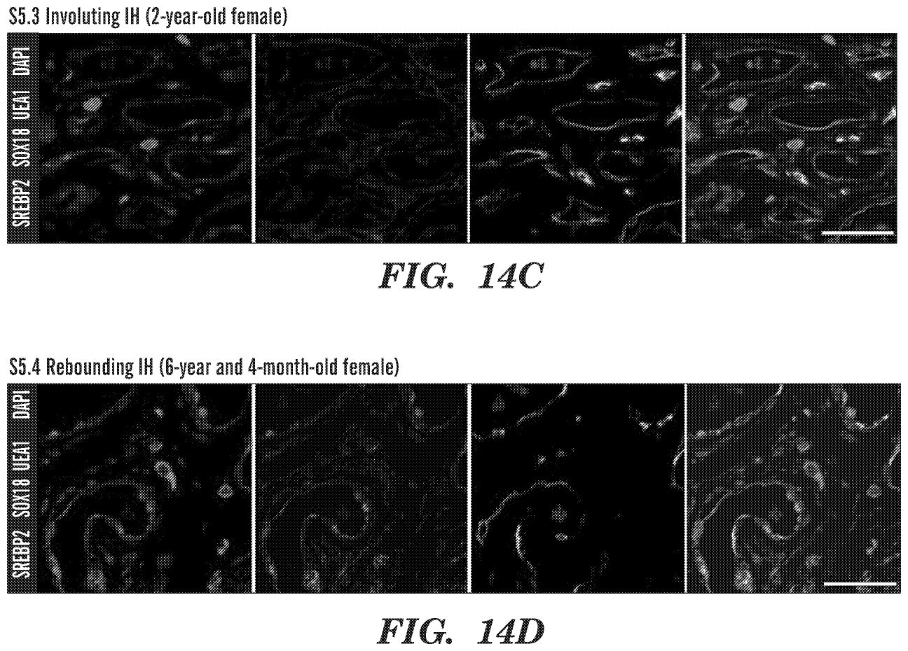

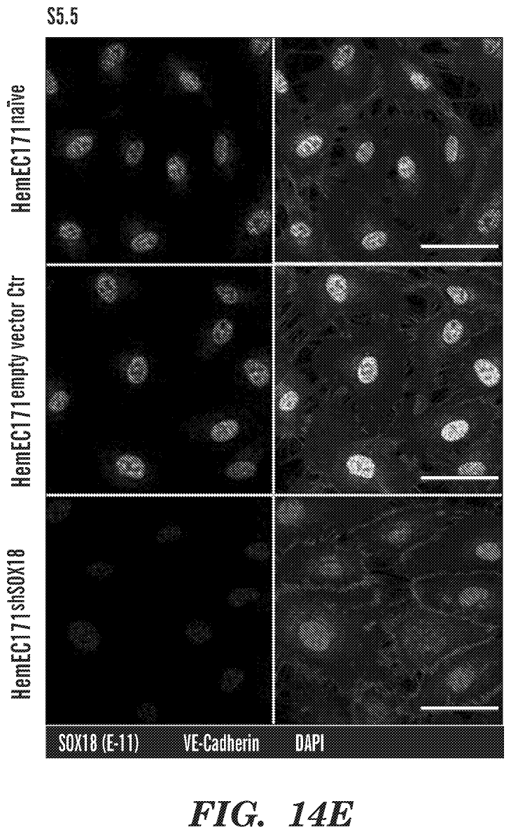

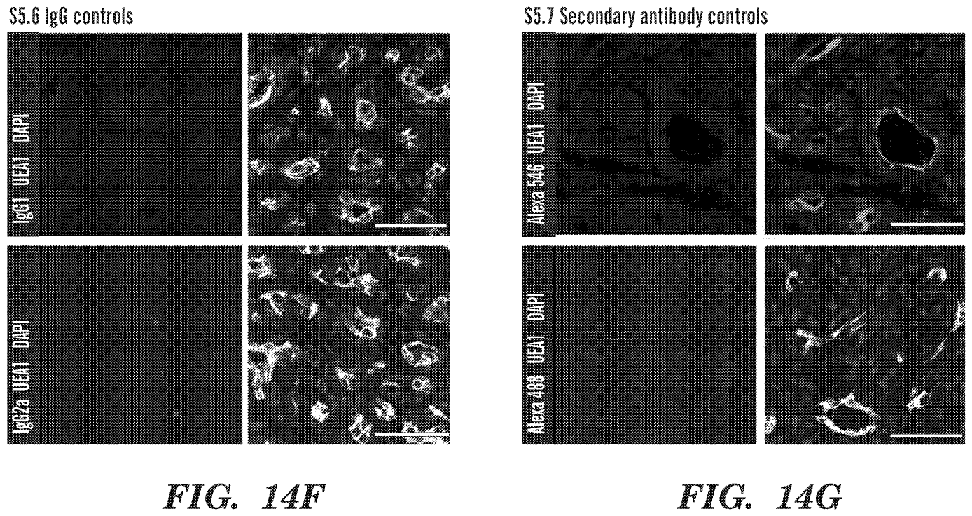

Based on the transcriptomic characterization from patient-derived HemSC, the activation of the MVP and SOX18 expression pattern was further investigated. This approach is to determine whether these molecular markers assist in defining a specific stage of IH progression that would respond to statin inhibition. Proliferating and involuting IH tissue sections (n=10) from specimens removed from children with IH who had not been treated with propranolol or corticosteroid (or any other treatment) before were stained. Immunofluorescent staining with antibodies against human SOX18, SREBP2 and with the lectin UEA1, which specifically binds to human endothelial cells, showed colocalization of SOX18 and SREBP2 in endothelial nuclei throughout the proliferating phase IH sections. Age-matched human skin (n=4), stained in parallel for comparison, was devoid of SOX18. Further, SOX18 was largely absent from vessels in involuting phase IH. SOX18 is not needed to maintain endothelial phenotype and is thus not routinely detected in mature, quiescent blood vessels³⁵. Next, SOX18+SREBP2+ cells/total endothelial nuclei were quantified as a metric for active MVP. SOX18+/SREBP2+ double positive cell nuclei in involuting phase IH specimens were significantly reduced compared to proliferating phase IH (FIGS. 13A, 13B, 13C, 14A-14C). In addition, tissue from two rebounding IH patients that were biopsied prior to them receiving treatment with propranolol were analyzed. Both rebounding IH were positive for nuclear expressed SOX18 and SREBP2 along the endothelium. Of note, histological features of proliferating IH (nuclear co-expression of SOX18 and SREBP2) as well as involuting IH (enlarged vessels, connective tissue with stromal cells and with adipocyte islands) in the rebounding IH were observed (FIGS. 13D, 14D). Quantification of SOX18+SREBP2+ cells/total endothelial nuclei in rebounding IH demonstrated significantly increased expression compared to skin controls and involuting IH and a similar expression compared to proliferating IH (FIG. 13E).

The SOX18 antibodies used throughout this study (D-8 and E-11) for Western Blot and immunofluorescent staining were validated in naïve HemEC andHemEC^{empty vector control} versus HemEC^{shSOX18} (FIG. 14E). Immunostaining of IH sections with respective isotype matched control IgG and secondary antibodies are shown in FIG. 14F-14G). In summary, the nuclear co-localization of SOX18 and SREBP2 in proliferating phase IH is a metric for MVP activity and may be predictive of responsiveness to statin therapy. Consequently, nuclear SOX18/SREBP2 colocalization in IH may serve as a theranostic tool for treatment indication and response prediction.

### Discussion:

In this study, the SOX18-MVP axis is demonstrated to be a central regulator of the pathogenesis of IH.

The R(+) enantiomer of propranolol, which was shown previously to disrupt SOX18 transcriptional activities, downregulated the expression of several genes in the MVP and reduced mature SREBP2 protein, both in a SOX18-dependent manner. R(+) propranolol or the SOX18 inhibitor Sm4 decreased new synthesis of cholesterol in HUVECs. It is contemplated herein that SOX18 augments the transcriptional control of MVP genes to regulate cholesterol and isoprenoid biosynthesis in HemSC undergoing endothelial differentiation. Of note, SOX18 levels increase over the course of HemSC to EC differentiation, and this corresponds to MVP sensitivity to R(+) propranolol. Furthermore, SOX18 ChIP binding sites are present in HMGCS1, HMGCR, and ABCA1 loci. The present experimental results and observations combine to demonstrate the SOX18-MVP axis in the etiology of IH, indicating statins are a new therapeutic option for IH.

Additional experiments support these new insights. Competitive inhibition of the rate-limiting enzyme of the MVP—HGMCR—with statins (simvastatin and atorvastatin) resulted in significantly reduced HemSC blood vessel formation in a dose-dependent manner with unaffected weight and glucose levels. Furthermore, the numbers of angiogenic mouse blood vessels in the HemSC/Matrigel implants were unaffected, indicating statins had little or no effect on host angiogenesis. Additional evidence for SOX18 regulation of the MVP was obtained when the RaOp SOX18 was expressed in HUVECs. The RaOp dominant negative SOX18, which is thought to “poison” normal SOX18 function³⁶, downregulated HMGCS1 and HMGCR. SOX18 and SREBP2 were found colocalized in endothelial nuclei in proliferating phase IH and 2 cases of rebounding IH but not in involuting phase IH or normal skin indicating the SOX18-SREBP2 may be restricted to nascent vessels in IH. Rebound of IH defies the classic IH life cycle; in these two cases, rebound occurred at 6 and 9 years of age. The nuclear co-localized SOX18 and SREBP2 in proliferating phase IH and rebounding IH suggests that SOX18⁺/SREBP2⁺ may serve as biomarkers for the vasculogenic capacity of the tumor. The nuclear expression of SOX18 and SREBP2 may give important biomolecular clues for patients to predict treatment response to statins and may thereby provide a novel theranostic approach.

SOX18 is a firmly established transcriptional regulator of vascular and lymphatic development, and tumor angiogenesis^{11-14,37,38}. The small molecule inhibitor of SOX18, Sm4, suppresses vascular development in zebrafish and both tumor angio- and lymph-angiogenesis in a breast cancer model^{29,37}. Notably, while glycolysis and fatty acid oxidation have been well established in physiological and pathological endothelial cell metabolism^{39,40}, the MVP has so far not been implicated. The instant discovery of a functional link between SOX18 and the MVP brings to light an entirely novel concept in endothelial differentiation and vasculogenesis. By interfering with SOX18 transcriptional activity and downstream regulation of the MVP, R+ propranolol acts as an EC phenotype-specific statin.

Without wishing to be bound by theory, the R(+) propranolol mediated reduction in MVP genes and cholesterol biosynthesis may exert effects on membrane fluidity and ruffling or activation of important signaling pathways by prenylation.

Pleiotropic benefits of statins, beyond their lowering effect on LDL-cholesterol levels in plasma, have been well documented and inferred to provide cardiovascular disease protection. These include inhibiting inflammatory response, increasing the bioavailability of nitric oxide, promoting re-reendothelialization, and reducing oxidative stress^{23,24,43}. The underlying mechanisms, however, remain elusive. A recent study by Liu et al. suggests epigenetic effects demonstrating that simvastatin significantly improves human induced pluripotent stem cell-derived endothelial cell function by reducing chromatin accessibility under physiological and pathological conditions²⁸. Moreover, it has long been established that statins block mammalian target of rapamycin (mTOR) and thereby inhibit AKT signaling⁴⁴, which is central to balancing vascular growth with endothelial quiescence. It was further shown that mTOR complex 1 reduces ER cholesterol levels which in turn activates SREBP2 and the MVP⁴⁵. Of note, PI3K-AKT-mTOR signaling is crucial in the pathogenesis of slow-flow malformations^{46,47}. Hence, statins are contemplated for treatment of these vascular anomalies as well.

Statins are among the most widely used drugs worldwide over the past 40 years. The resulting reduction in cholesterol synthesis consequently increases LDL-receptor expression and this in turn clears low-density lipoprotein cholesterol from the circulation, resulting in cardiovascular benefits. Although they are generally considered a safe and well tolerated drug class, they are associated with an increased risk of muscle pain, diabetes mellitus and hepatic transaminase elevations⁴⁸. Addressed herein are potential side effects by measuring glucose levels and body weight in the statin treated xenograft mice. An increase or decrease in glucose levels or weight changes with simvastatin and atorvastatin were not observed over the treatment course of 7 days. Bearing in mind drug safety as the highest priority when considering translating statins to infants, we performed a dose response experiment of simvastatin. The lowest dose that showed significant reduction in IH vessels in the xenograft model was 1 mg/kg/d. This translates to a human equivalent dose of 0.081 mg/kg/d⁴⁹ and is thus 6.25× below the recommended dose of systemic simvastatin in infants, which starts at 0.5 mg/kg/d for other treatment indications. Further, topical versus systemic application of statins in infants with less complex IH may increase safety as.

It is contemplated herein that the novel link between SOX18 and the MVP supports statin treatment various pathophysiological conditions of the endothelium, especially other types of vascular overgrowth or vascular anomalies. In contrast with other vascular anomalies for which genetic drivers have been identified in recent years, the genetic drivers of IH are unknown. While the genetics have facilitated improved targeted therapeutic approaches^{17,18}, critical molecular players in IH have been uncovered by studying mechanisms of action of serendipitously discovered drugs^{9,10,15,16}. This study demonstrates a mechanistic, SOX18-dependent link between beta blockers and statins.

In summary, described herein is a novel SOX18-MVP axis as a central molecular driver for HemSC vasculogenesis. Experiments in a xenograft model demonstrate that simvastatin or atorvastatin inhibit IH vessel formation, demonstrating statins can be repurposed to treat this common benign vascular tumor of infancy, topically or systemically. Moreover, demonstrated herein is the suppressive effect of R(+) propranolol on the transcription of MVP genes via SOX18. This provides a mechanistic link between beta blockers and statins.

### Methods:

Cell isolation and culturing: The clinical diagnosis of IH was confirmed in the Department of Pathology of Boston Children's Hospital. Single-cell suspensions prepared from proliferating and involuting IH specimens were deidentified and designated as specified in FIG. 6. HemSCs or HemECs were selected using anti-CD133- and anti-CD31-coated magnetic beads (Miltenyi Biotec), respectively, and expanded. Testing for mycoplasma contamination by qPCR was performed when cells were thawed and every 4-6 weeks thereafter. Cells were cultured on fibronectin-coated (0.1 μg/cm²; MilliporeSigma) plates with Endothelial Growth Medium-2 (EGM-2; Lonza), which consists of Endothelial Cell Growth Basal Medium-2 (EBM-2; Lonza), SingleQuot supplements (all except hydrocortisone and gentamicin; Lonza), 10% heat-inactivated FBS (Hyclone), and 1×GPS (292 mg/mL glutamine, 100 U/mL penicillin, 100 mg/mL streptomycin; Mediatech). Cells were cultured at 37° C. in a humidified incubator with 5% CO₂.

Hemangioma endothelial differentiation assay: To induce endothelial differentiation, HemSCs (types 125, 149, 150, 165, 167, 171) were seeded on fibronectin-coated plates at a density of 20,000 cells/cm² in EGM2. After 18-24 hours, cells were starved for 16 hours in 2% BSA/serum-free EGM2. The medium was replaced with serum-free EBM-2 containing 10 ng/mL VEGF-B (R&D Systems), 1× insulin transferrin-selenium, 1:5000 linoleic acid-albumin, 1 mM dexamethasone, and 60 mM ascorbic acid-2-phosphate (68). A stock solution of 10 mM propranolol hydrochloride (MilliporeSigma), R(+) propranolol hydrochloride (MilliporeSigma), atenolol (MilliporeSigma), and R(+) atenolol (MilliporeSigma) was prepared in DMSO (MilliporeSigma). Cells were cultured in the VEGF-B, serum-free media to undergo endothelial differentiation for 6 days. 20 μM R+ propranolol was added and incubated for two hours and cells (n=6) harvested for RNA sequencing (FIGS. 2A-2C). Continuous as well as short term treatment with with 20 μM R+ propranolol was applied to validate the RNA sequencing data of HemSC (n=3) undergoing endothelial differentiation by qPCR (FIGS. 2D, 2E). Continuous treatment with 1 μM simvastatin, 0.1 μM atorvastatin as well as 20 μM R+ propranolol as a positive control was applied to differentiating HemSC (n=3) to demonstrate the inhibitory effect of statins in vitro (FIG. 9A). Respective vehicle controls of each drug were used, whereby vehicle without VEGF-B served as a negative control and DMSO with VEGF-B as a positive control. The toxicity of simvastatin and atorvastatin was tested by counting HemSC (n=3) treated with simvastatin, atorvastatin, or vehicle over 6 days in endothelial differentiation medium (FIG. 10A) or in normal growth medium for 24 hours (FIG. 10B) relative to respective vehicle controls.

Cell culture: HUVECs were grown in EBM-2 media (Lonza, CC-3156) and supplemented with EGM-2 bullet kit (Lonza, CC-3162). HUVECs were seeded on 0.5% gelatin-coated 6-well plates at a density of 1.5×10⁵ cells/well for overnight.

Methyl-β-cyclodextrin treatment: The day after cell seeding cells were treated with, (MBCD, Sigma #C4555) at a concentration of 2.5 mM to ensure a similar level of endogenous cholesterol among the groups. The treatment with MBCD was completely withdrawn after 4 hours by discarding the media and washed the cells with PBS. After the PBS wash cells were treated with either PBS, 20 μM R+ propranolol hydrochloride (Sigma-Aldrich, Cat #P0689), DMSO, or 40 μM of Sm4 (Sigma-Aldrich, SML1999) for 18 hours. Cells were then washed with PBS and trypsinized. Following trypsinization cells were washed with PBS and centrifuged (FIG. 4B, 4C).

Cholesterol measurement using Targeted Mass Spectrometry: Lipids were extracted from the cell pellets through one-phase extraction with 202 μL of BuOH: MeOH (1:1) (Sigma #71-36-3 and #67-56-1) that contains 10 μmol of cholesterol-d7 (Cayman Chemical #83199-47-7) as the internal standard. The cholesterol analysis was performed on a Thermo Fisher TSQ Altis triple quadrupole mass spectrometer, operated in positive ion mode, coupled to a Vanquish UHPLC system (Thermo Fisher, Waltham, USA) using the transition from precursor mass of m/z 369.3516 (MS1) to m/z 161.1 (MS3) for cholesterol and 376.4 to 161.1 for cholesterol-d7 [REF]. The solvent pair includes solvent A (100% H2O, 0.1% FA and 2 mM NH₄COO) and solvent B (100% MeOH, 0.1% FA and 2 mM NH₄COO⁻) with a flow rate at 0.3 mL/min (80% B at the start). Cholesterol and its deuterated standard were separated on an Agilent Eclipse Plus C8 column, and peaks were integrated with TraceFinder 5.1 (Thermo Fisher, Waltham, USA) using the daughter ion at m/z 161.1 (FIG. 4B, 4C).

Transfection and flow cytometry: The day after cell seeding transfection was performed using X-tremeGene HP Transfection Reagent kit (Roche) to introduce 500 ng of Halo-RaOp DNA using EBM-2 culture media without antibiotic. Cells were incubated at 37 C with 5% overnight. The next day cells incubated with 5 nM of JF646-Halo-ligand for 15 min. Then washed with PBS and trypsonzied. Cells were then spun down and washed with PBS and permeablized with 0.2% Triton X-100 for 10 min, then washed and blocked with 5% BSA/PBS for 1 hr. Cells were then labelled with 1:300 dilution of anti-human HMGCR (ThermoFisher, PA537367) or HMGCS1 (ThermoFisher, PA513604) for 1 hour in at room temp in the dark. Cells were then washed with PBS and stained with 1:500 dilution of anti-rabbit IgG (Invitrogen A11008) for 30 min then washed with PBS. After the final wash the cells were then imaged on a BD Fortessa X-20. Data analysis was performed using FlowJo software (FIG. 4D, 4E).

shRNA knockdown of SOX18: HemEC (n=3) were transduced with SOX18 shRNA lentivirus (TRCN0000017450, Sigma) or an empty vector control virus (Sigma) followed by puromycin (1 μg/mL) selection for 5 days. Thereafter, cells were maintained in full media without puromycin. Knockdown efficiency was confirmed by qPCR and Western Blot (FIG. 7C-7G, 8A-8B).

Western Blot: Cells were seeded on fibronectin-coated plates at a density of 20,000 cells/cm² in full growth media for 30 hours followed by treatment with 20 μM R+ propranolol or PBS for 2 hours. Cells were lysed in a RIPA-based lysis buffer (25 mM Tris HCl pH 7.6, 150 mM NaCl, 1% NP-40, 1% sodium deoxycholate, 0.1% SDS) with protease and phosphatase inhibitors (Cell Signaling Technology). Cell extracts were electrophoresed by sodium dodecyl sulfate-polyacrylamide gel electrophoresis (SDS-PAGE), transferred to nitrocellulose or to polyvinylidene difluoride (PDVF) and probed with SOX18 D-8 (Santa Cruz Biotechnology) or SREBP2 clone 22D5 (MilliporeSigma) and GAPDH (Cell Signaling Technology). All signals were detected by enhanced chemiluminescence. The densitometric analysis was conducted by using Fiji ImageJ software.

In vivo murine model for human blood vessel formation: A stock solution simvastatin (105 mM; MilliporeSigma) or atorvastatin (10 mM; MilliporeSigma) was prepared in DMSO. The stock solution was diluted with PBS to the indicated treatment concentrations. Experiments were carried out with 3×10⁶ HemSCs per implant. In vivo, the HemSCs undergo vasculogenesis and form anastomoses with ingrowing host vessels; the implants do not expand in size. HemSCs (n=4, types 125, 147, 149, 150) were grown in EGM2 medium until 90% confluency. Twenty-four hours before harvesting, 5 μM treatment with simvastatin or 0.5 μM treatment or DMSO as a control was added to the media. Cells were counted after the 24-hour pretreatment and suspended in 200 μL Matrigel (Corning) preadjusted with 1 μg/mL basic FGF (bFGF) (ProSpec), 1 μg/mL erythropoietin (EPO) (ProSpec), and 2.5 μM (simvastatin) or 0.25 μM (atorvastatin) drug or PBS with max (0.005% DMSO) on ice. The Matrigel/cell suspensions were injected subcutaneously into the flanks of 6-week-old male athymic nu/nu mice (envigo), placing 2 implants per mouse (n=3-5 mice/group; FIG. 9B). The mice were treated with 0.1-50 mg/kg simvastatin or 1-15 mg/kg or DMSO (maximal concentration 11.4%) as a control (200 μL/mouse, i.p.) twice a day. Blood glucose levels of the mice were measured daily before the morning i.p. injection. Glucose concentrations were measured in tail vein blood using the One Touch UltraSmart Blood Glucose Monitoring System (LifeScan). Body weight was measured before the injections (day 0), on day 4 and before removal of the implants (day 8). After 7 days, the mice were euthanized and the implants were removed, fixed in formalin, embedded in paraffin, and analyzed by H&E staining and IF. Blood vessels (indicated by the luminal structures containing 1 or more red blood cells) and CD31⁺ human vessels were counted in 5 fields/section, 2 sections/implant. Each field was 425.1 μm×425.1 μm=0.18071 mm2, and sections were from the middle of the implant. Vessel density is expressed as vessels/mm².

Histology and IF analysis: FFPE tissue sections (5 μm) of the Matrigel implants were deparaffinized and either directly stained with H&E or immersed in an antigen retrieval solution (citrate-EDTA buffer containing 10 mM citric acid, 2 mM EDTA, and 0.05% Tween-20, pH 6.2) for 20 minutes at 95° C.-99° C. Sections were subsequently blocked for 30 minutes in TNB Blocking Buffer (PerkinElmer) followed by incubation with human-specific CD31 monoclonal antibody (1:30, mouse anti-human; Dako, Glostrup, 0823) to stain human ECs. Next, sections were incubated with Alexa Fluor 647 chicken anti-mouse IgG (1:200, Invitrogen, Thermo Fisher Scientific) as a secondary antibody (FIG. 9C, 9D).

Mouse-specific CD31 monoclonal antibody (1:100, R&D Systems) was used to differentiate mouse vessels in the Matrigel implants. As a secondary antibody, a Alexa Fluor 647 chicken anti-goat IgG was applied (FIG. 10D). Tissue specificity of the anti-human and anti-mouse antibody was confirmed by negative staining in mouse lung and human skin tissue, respectively (FIG. 10E).

FFPE tissue sections (5 μm) from patients with IH were deparaffinized, immersed in an antigen retrieval solution, and blocked for 30 minutes in 10% donkey serum followed by incubation with mouse anti-human SOX18 (1:50; Santa Cruz Biotechnology), rabbit anti-human SREBP2 and UEA1 fluorescently labeled with Alexa Fluor 649 (1:50; Vector Laboratories). Next, the sections were incubated with Alexa Fluor 488 donkey anti-mouse IgG and Alexa Fluor 546 donkey anti-rabbit IgG (both 1:200; Invitrogen, Thermo Fisher Scientific) as secondary antibodies. All slides were mounted using DAPI (Molecular Probes, R37606) to visualize nuclei. IF Images were acquired by a Zeiss Airyscan LSM 880 Fast confocal microscope. Images were analyzed through a 20 or 63× objective lens using a Zeiss Axio Imager M1 microscope (Carl Zeiss Microscopy). All images were analyzed using Fiji ImageJ software (FIG. 13).

Cell staining: HemEC171^{naïve}, HemEC171^{empty vector Ctr} and HemEC171^{shSox18} were seeded on fibronectin-coated plates at a density of 20,000 cells/cm² in full growth media for 30 hours on 2 cm² slides, fixed in 4% PFA and blocked in 5% BSA/0.3% Triton x-100 for 1 hour. Next primary antibodies for mouse anti-human SOX18 (1:100, Santa Cruz) and rabbit anti-human VE-Cadherin (1:100, R&D Systems) were applied followed by secondary antibody incubation with Alexa Fluor 488 donkey anti-mouse IgG and Alexa Fluor 546 donkey anti-rabbit IgG (both 1:200; Invitrogen, Thermo Fisher Scientific). Slides were mounted using DAPI (Molecular Probes, R37606) to visualize nuclei (FIG. 14A-14G).

RNA isolation and qPCR: Total cellular RNA was extracted from cells with a RNeasy Micro Extraction Kit (QIAGEN). Reverse transcriptase reactions were performed using an iScript cDNA Synthesis Kit (Bio-Rad). qPCR was performed using Kapa SYBR FAST ABI Prism 2×qPCR Master Mix (Kapa BioSystems). Amplification was carried out in a StepOne Real-Time PCR System (Applied Biosystems). A relative standard curve for each gene amplification was generated to determine the amplification efficiency, with greater than 90% considered acceptable. Fold increases in gene expression were calculated according to the ΔΔCt method, with each amplification reaction performed in duplicate or triplicate (REF). Gene expression was normalized to the PBS treatment. ATP5B was used as housekeeping gene expression reference.

Statistics: Data were analyzed and plotted using GraphPad Prism 10.1.0 (GraphPad Software). Results are displayed as the mean±SD unless otherwise indicated. For experiments in which cells were treated with alternative drugs, the differences were assessed by 1-way ANOVA followed by Bonferroni's or Tukey's post hoc test for multiple comparisons of different treatment modalities or Dunnett's test for multiple comparisons to compare every treatment mean with that of the vehicle control. For comparisons between treatment and control groups, 2-tailed, unpaired Student t tests were applied. Significance was assessed by either Welch's t test or a 2-way ANOVA with Šidák multiple-comparison correction.

### REFERENCES

1. Leaute-Labreze, C., Harper, J. I. & Hoeger, P. H. Infantile haemangioma. Lancet 390, 85-94 (2017).
2. Leaute-Labreze, C., et al. Propranolol for severe hemangiomas of infancy. N Engl J Med 358, 2649-2651 (2008).
3. Leaute-Labreze, C., et al. A randomized, controlled trial of oral propranolol in infantile hemangioma. N Engl J Med 372, 735-746 (2015).
4. Leaute-Labreze, C., et al. Safety of Oral Propranolol for the Treatment of Infantile Hemangioma: A Systematic Review. Pediatrics 138 (2016).
5. Pope, E. Commentary: Beta-blockers and sleep problems. Pediatr Dermatol 38, 378-379 (2021).
6. Hermans, M. M., et al. Long-term neurocognitive functioning of children treated with propranolol or atenolol for infantile hemangioma. Eur J Pediatr 182, 757-767 (2023).
7. Mehvar, R. & Brocks, D. R. Stereospecific pharmacokinetics and pharmacodynamics of beta-adrenergic blockers in humans. J Pharm Pharm Sci 4, 185-200 (2001).
8. Khan, Z. A., et al. Multipotential stem cells recapitulate human infantile hemangioma in immunodeficient mice. Journal of Clinical Investigation 118, 2592-2599 (2008).
9 Overman, J., et al. R-propranolol is a small molecule inhibitor of the SOX18 transcription factor in a rare vascular syndrome and hemangioma. Elife 8 (2019).
10. Seebauer, C. T., et al. Non-beta blocker enantiomers of propranolol and atenolol inhibit vasculogenesis in infantile hemangioma. J Clin Invest 132 (2022).
11. Patel, J., et al. Functional Definition of Progenitors Versus Mature Endothelial Cells Reveals Key SoxF-Dependent Differentiation Process. Circulation 135, 786-805 (2017).
12. Herpers, R., van de Kamp, E., Duckers, H. J. & Schulte-Merker, S. Redundant roles for sox7 and sox18 in arteriovenous specification in zebrafish. Circ Res 102, 12-15 (2008).
13. Francois, M., et al. Sox18 induces development of the lymphatic vasculature in mice. Nature 456, 643-647 (2008).
14. Young, N., et al. Effect of disrupted SOX18 transcription factor function on tumor growth, vascularization, and endothelial development. J Natl Cancer Inst 98, 1060-1067 (2006).
15. Greenberger, S., Boscolo, E., Adini, I., Mulliken, J. B. & Bischoff, J. Corticosteroid suppression of VEGF-A in infantile hemangioma-derived stem cells. N Engl J Med 362, 1005-1013 (2010).
16. Greenberger, S., et al. Rapamycin suppresses self-renewal and vasculogenic potential of stem cells isolated from infantile hemangioma. J Invest Dermatol 131, 2467-2476 (2011).
17. Queisser, A., Seront, E., Boon, L. M. & Vikkula, M. Genetic Basis and Therapies for Vascular Anomalies. Circ Res 129, 155-173 (2021).
18. Dompmartin, A., et al. The VASCERN-VASCA Working Group Diagnostic and Management Pathways for Venous Malformations. J Vasc Anom (Phila) 4, e064 (2023).
19. Zhang, C., Jin, D. D., Wang, X. Y., Lou, L. & Yang, J. Key Enzymes for the Mevalonate Pathway in the Cardiovascular System. J Cardiovasc Pharmacol 77, 142-152 (2021).
20. Horton, J. D., Goldstein, J. L. & Brown, M. S. SREBPs: activators of the complete program of cholesterol and fatty acid synthesis in the liver. J Clin Invest 109, 1125-1131 (2002).
21. Yamauchi, Y., et al. Deficiency in the Lipid Exporter ABCA1 Impairs Retrograde Sterol Movement and Disrupts Sterol Sensing at the Endoplasmic Reticulum. J Biol Chem 290, 23464-23477 (2015).
22. Chou, R., Dana, T., Blazina, I., Daeges, M. & Jeanne, T. L. Statins for Prevention of Cardiovascular Disease in Adults: Evidence Report and Systematic Review for the US Preventive Services Task Force. JAMA 316, 2008-2024 (2016).
23. Laufs, U., La Fata, V., Plutzky, J. & Liao, J. K. Upregulation of endothelial nitric oxide synthase by HMG CoA reductase inhibitors. Circulation 97, 1129-1135 (1998).
24. Sen-Banerjee, S., et al. Kruppel-like factor 2 as a novel mediator of statin effects in endothelial cells. Circulation 112, 720-726 (2005).
25. Greenwood, J. & Mason, J. C. Statins and the vascular endothelial inflammatory response. Trends Immunol 28, 88-98 (2007).
26. Karlic, H., et al. Inhibition of the mevalonate pathway affects epigenetic regulation in cancer cells. Cancer Genet 208, 241-252 (2015).
27. Tikoo, K., et al. Tissue specific up regulation of ACE2 in rabbit model of atherosclerosis by atorvastatin: role of epigenetic histone modifications. Biochem Pharmacol 93, 343-351 (2015).
28. Liu, C., et al. Statins improve endothelial function via suppression of epigenetic-driven EndMT. Nat Cardiovasc Res 2, 467-485 (2023).
29. Fontaine, F., et al. Small-Molecule Inhibitors of the SOX18 Transcription Factor. Cell Chem Biol 24, 346-359 (2017).
30. Pennisi, D., et al. Mutations in Sox18 underlie cardiovascular and hair follicle defects in ragged mice. Nat Genet 24, 434-437 (2000).
31. Radhakrishnan, A., Goldstein, J. L., McDonald, J. G. & Brown, M. S. Switch-like control of SREBP-2 transport triggered by small changes in ER cholesterol: a delicate balance. Cell Metab 8, 512-521 (2008).
32. McFarlane, M. R., et al. Scap is required for sterol synthesis and crypt growth in intestinal mucosa. J Lipid Res 56, 1560-1571 (2015).
33. Chen, L., et al. Endogenous sterol intermediates of the mevalonate pathway regulate HMGCR degradation and SREBP-2 processing. J Lipid Res 60, 1765-1775 (2019).
34. Wassif, C. A., et al. A placebo-controlled trial of simvastatin therapy in Smith-Lemli-Opitz syndrome. Genet Med 19, 297-305 (2017).
35. Downes, M. & Koopman, P. SOX18 and the transcriptional regulation of blood vessel development. Trends Cardiovasc Med 11, 318-324 (2001).
36. McCann, A. J., et al. A dominant-negative SOX18 mutant disrupts multiple regulatory layers essential to transcription factor activity. Nucleic Acids Res 49, 10931-10955 (2021).
37. Overman, J., et al. Pharmacological targeting of the transcription factor SOX18 delays breast cancer in mice. Elife 6 (2017).
38. Donovan, P., et al. Endovascular progenitors infiltrate melanomas and differentiate towards a variety of vascular beds promoting tumor metastasis. Nat Commun 10, 18 (2019).
39. Eelen, G., et al. Endothelial Cell Metabolism. Physiol Rev 98, 3-58 (2018).
40. Zeng, Q., et al. Understanding tumour endothelial cell heterogeneity and function from single-cell omics. Nat Rev Cancer 23, 544-564 (2023).
41. Palsuledesai, C. C. & Distefano, M. D. Protein prenylation: enzymes, therapeutics, and biotechnology applications. ACS Chem Biol 10, 51-62 (2015).
42. Hu, W., et al. NOGOB receptor-mediated RAS signaling pathway is a target for suppressing proliferating hemangioma. JCI Insight 6 (2021).
43. Wolfrum, S., Jensen, K. S. & Liao, J. K. Endothelium-dependent effects of statins. Arterioscler Thromb Vasc Biol 23, 729-736 (2003).
44. Lashgari, N. A., et al. Statins block mammalian target of rapamycin pathway: a possible novel therapeutic strategy for inflammatory, malignant and neurodegenerative diseases. Inflammopharmacology 31, 57-75 (2023).
45. Eid, W., et al. mTORC1 activates SREBP-2 by suppressing cholesterol trafficking to lysosomes in mammalian cells. Proc Natl Acad Sci USA 114, 7999-8004 (2017).
46. Seront, E., Van Damme, A., Boon, L. M. & Vikkula, M. Rapamycin and treatment of venous malformations. Curr Opin Hematol 26, 185-192 (2019).
47. Van Damme, A., Seront, E., Dekeuleneer, V., Boon, L. M. & Vikkula, M. New and Emerging Targeted Therapies for Vascular Malformations. Am J Clin Dermatol 21, 657-668 (2020).
48. Cholesterol Treatment Trialists, C. Effect of statin therapy on muscle symptoms: an individual participant data meta-analysis of large-scale, randomised, double-blind trials. Lancet 400, 832-845 (2022).
49. Nair, A. B. & Jacob, S. A simple practice guide for dose conversion between animals and human. J Basic Clin Pharm 7, 27-31 (2016).
50. Jira, P. E., et al. Simvastatin. A new therapeutic approach for Smith-Lemli-Opitz syndrome. J Lipid Res 41, 1339-1346 (2000).
51. Chan, Y. M., et al. Effects of dietary cholesterol and simvastatin on cholesterol synthesis in Smith-Lemli-Opitz syndrome. Pediatr Res 65, 681-685 (2009).
52. Svoboda, M. D., Christie, J. M., Eroglu, Y., Freeman, K. A. & Steiner, R. D. Treatment of Smith-Lemli-Opitz syndrome and other sterol disorders. Am J Med Genet C Semin Med Genet 160 C, 285-294 (2012).
53. Christiansen, A. G., Koppelhus, U. & Sommerlund, M. Skin Abnormalities in CHILD Syndrome Successfully Treated with Pathogenesis-based Therapy. Acta Derm Venereol 95, 752-753 (2015).
54. Cervantes, J., Jimenez, J. J., DelCanto, G. M. & Tosti, A. Treatment of Alopecia Areata with Simvastatin/Ezetimibe. J Investig Dermatol Symp Proc 19, S25-S31 (2018).
55. Shin, J. M., et al. Putative therapeutic mechanisms of simvastatin in the treatment of alopecia areata. J Am Acad Dermatol 84, 782-784 (2021).
56. Albinana, V., et al. Propranolol: A “Pick and Roll” Team Player in Benign Tumors and Cancer Therapies. J Clin Med 11 (2022).
57. Poynter, J. N., et al. Statins and the risk of colorectal cancer. N Engl J Med 352, 2184-2192 (2005).
58. Nielsen, S. F., Nordestgaard, B. G. & Bojesen, S. E. Statin use and reduced cancer-related mortality. N Engl J Med 367, 1792-1802 (2012).
59. Mamtani, R., et al. Disentangling the Association between Statins, Cholesterol, and Colorectal Cancer: A Nested Case-Control Study. PLOS Med 13, e1002007 (2016).
60. Voorneveld, P. W., et al. Statin Use After Diagnosis of Colon Cancer and Patient Survival. Gastroenterology 153, 470-479 e474 (2017).
61. Li, Y., He, X., Ding, Y., Chen, H. & Sun, L. Statin uses and mortality in colorectal cancer patients: An updated systematic review and meta-analysis. Cancer Med 8, 3305-3313 (2019).
62. Pereira, M., Matuszewska, K., Glogova, A. & Petrik, J. Mutant p53, the Mevalonate Pathway and the Tumor Microenvironment Regulate Tumor Response to Statin Therapy. Cancers (Basel) 14 (2022).
63. Scott, O. W., et al. Post-diagnostic statin use and breast cancer-specific mortality: a population-based cohort study. Breast Cancer Res Treat 199, 195-206 (2023).

1. A method of treating a vascular anomaly in a subject in need thereof, the method comprising administering a composition comprising at least one statin to the subject.

2. The method of claim 1, wherein the statin is selected from the group consisting of:
simvastatin; atorvastatin; cerivastatin; fluvastatin; lovastatin; mevastatin; pitavastatin;
pravastatin; and rosuvastatin.

3. The method of claim 1, wherein the statin is simvastatin.

4. The method of claim 1, wherein the statin is atorvastatin.

5. The method of claim 1, wherein the vascular anomaly is selected from the group consisting of:
infantile hemangioma, congenital hemangioma, pyogenic granuloma, tufted angioma, kaposiform hemangioendothelioma, epitheloid hemangioendothelioma (EHE), capillary malformations, arteriovenous malformations, venous malformations, lymphatic malformations, complex lymphatic malformations, generalized lymphatic anomaly, central conducting lymphatic anomaly, kaposiform lymphangiomatosis, and Gorham-Stout disease.

6. The method of claim 1, wherein the vascular anomaly is infantile hemangioma.

7. The method of claim 1, wherein the subject is further administered at least one beta-blocker, mTOR inhibitor, and/or steroid.

8. The method of claim 7, wherein the beta-blocker is selected from the group consisting of:
R+ atenolol; atenolol; nadolol; propranolol; R+propranolol; and timolol.

9. The method of claim 7, wherein the beta-blocker is R+ propranolol.

10. The method of claim 7, wherein the mTOR inhibitor is selected from the group consisting of:
sirolimus; everolimus; temisrolimus; and rapamycin.

11. The method of claim 7, wherein the mTOR inhibitor is rapamycin.

12. The method of claim 7, wherein the steroid is a corticosteroid.

13. The method of claim 12, wherein the corticosteroid is selected from the group consisting of:
dexamethasone; prednisone; prednisolone; triamcinolone; clobetasol propionate;
betamethasone valerate; betamethasone dipropionate; and mometasone furoate.

14. A combination or kit comprising:
at least one statin; and
at least one beta-blocker, mTOR inhibitor, and/or steroid.

15.-16. (canceled)

17. The combination or kit of claim 14, wherein the statin is selected from the group consisting of:
simvastatin; atorvastatin; cerivastatin; fluvastatin; lovastatin; mevastatin; pitavastatin;
pravastatin; and rosuvastatin.

18. The combination or kit of claim 14, wherein the statin is simvastatin.

19. The combination or kit of claim 14, wherein the statin is atorvastatin.

20. The combination or kit of claim 14, wherein the beta-blocker is selected from the group consisting of:
atenolol; nadolol; propranolol; R+ propranolol; and timolol.

21. (canceled)

22. The combination or kit of claim 14, wherein the mTOR inhibitor is selected from the group consisting of:
sirolimus; everolimus; temisrolimus; and rapamycin.

23.-24. (canceled)

25. The combination or kit of claim 14, wherein the steroid is a corticosteroid selected from the group consisting of:
dexamethasone; prednisone; prednisolone; triamcinolone; clobetasol propionate;
betamethasone valerate; betamethasone dipropionate; and mometasone furoate.

26.-29. (canceled)
